(19) United States
(12) Patent Application Publication
(10) Pub. No.: US 2023/0181658 A1
(43) Pub. Date: Jun. 15, 2023

(54) PROBIOTIC PREPARATION BASED ON W1/O/W2 TYPE DUAL EMULSION STRUCTURE, AND PREPARATION METHOD THEREFOR AND USE THEREOF

(71) Applicant: HUAZHONG AGRICULTURAL UNIVERSITY, Wuhan City (CN)

(72) Inventors: Yijie Chen, Wuhan (CN); Jinyu Chen, Wuhan (CN); Qi Zhou, Wuhan (CN); Beixi Li, Wuhan (CN); Qian Shen, Wuhan (CN); Wei Zheng, Wuhan (CN); Yilun Luo, Wuhan (CN); Shuang Ding, Wuhan (CN); Fei Han, Wuhan (CN); Liuqing Yang, Wuhan (CN)

(21) Appl. No.: 17/994,162

(22) Filed: Feb. 13, 2023

Related U.S. Application Data
(63) Continuation of application No. PCT/CN2021/094769, filed on May 20, 2021.

(30) Foreign Application Priority Data
May 26, 2020 (CN) 202010456385.8
May 6, 2021 (CN) 202110490478.7

(51) Int. Cl. A61K 35/747; A61K 9/113; A61K 47/24; A61K 9/00; A61K 47/34

(52) U.S. Cl. CPC A61K 35/747; A61K 9/0095; A61K 9/113; A61K 47/24; A61K 47/34

(57) ABSTRACT

A method for preparing a probiotic preparation based on a W₁/O/W₂ double emulsion structure includes steps of: using a solution containing probiotics or a mixed solution of probiotics and a probiotic protective agent as an internal water phase W₁; dissolving an emulsifier in a lipid phase O, and mixing the lipid phase O with the internal water phase W₁; then preparing a primary emulsion W₁/O by stirring and low-energy emulsification or high-energy emulsification; and using an emulsifier-contained solution as an external water phase W₂, and adding the external water phase W₂ to the primary emulsion W₁/O; then preparing a W₁/O/W₂ double emulsion by stirring and the low-energy emulsification or the high-energy emulsification, thereby obtaining the probiotic preparation. The double emulsion system is used to embed the probiotics, which can keep the probiotics active during low-temperature storage and freezing and thawing.

### CROSS REFERENCE OF RELATED APPLICATION

The application is a continuation application of a PCT application No. PCT/CN2021/094769, filed on May 20, 2021; and claims the priority of Chinese Patent Application No.CN202010456385.8, filed to the China National Intellectual Property Administration (CNIPA) on May 26, 2020, and of Chinese Patent Application No.CN202110490478.7, filed to the China National Intellectual Property Administration (CNIPA) on May 06, 2021, the entire content of which are incorporated hereby by reference.

### BACKGROUND OF THE PRESENT INVENTION

### Field of Invention

The present invention relates to a field of biotechnology, and more particularly to a probiotic preparation based on a W₁/O/W₂ double emulsion structure, and a preparation method therefor and a use thereof.

### Description of Related Arts

Probiotics are a type of active microorganisms that are beneficial to the host. They are a general term for active beneficial microorganisms that colonize the human intestinal tract and reproductive system and produce definite health effects to improve the host’s micro-ecological balance and apply beneficial effects. A large number of studies have shown that probiotics have a variety of physiological activities, such as: regulating intestinal health, improving the synthesis and bioavailability of nutrients in the body, lowering cholesterol levels, lowering blood pressure, alleviating tumors, preventing intestinal cancer, improving the body’s immunity, etc.

Products made of probiotics, also known as probiotic preparations, usually adopt representative strains such as Lactobacillus plantarum, Lactobacillus rhamnosus, Lactobacillus acidophilus, Lactobacillus reuteri, Lactobacillus bulgaricus, Bifidobacterium, Bifidobacterium bifidum, Bifidobacterium adolescentis and Streptococcus thermophilus. Studies have shown that the minimum intake of probiotics must reach 6-7log/mL to fully exert their physiological effects, which requires probiotic products to remain stable during storage and digestion.

However, most probiotics are facultatively anaerobic, and can be easily affected by oxygen during processing and storage. Under the influence of environmental pH and temperature, the storage stability of probiotics decreases. After oral ingestion, the probiotics enter the digestive tract, and will be further inactivated by gastric juice, digestive enzymes, and bile salts. Finally, the number of viable probiotics that colonizes the intestinal tract is usually lower than the theoretical minimum value that can be physiologically useful.

Conventionally, there are still many deficiencies in the probiotic products produced in China, such as unstable product quality and poor storage stability of probiotics. As a result, the number of viable probiotics in probiotic products decreases sharply in a short period of time during repeated freezing and thawing and storage, and the effective viable probiotic dosage cannot be guaranteed. In order to ensure the survival rate of probiotics, maintain their physiological activity, and improve the storage stability of probiotic products during the shelf life, the microencapsulation technology of probiotics and derivatives thereof have been widely studied all over the world. In these microencapsulation technologies, the survival rate and stability of probiotics mainly depend on the concentration and type of bacteria, the type of microcapsule wall material, and the outlet temperature of the spray dryer. Due to the complex influencing factors and complicated preparing processes, part of the probiotic will be inevitably inactivated. Furthermore, capsules need a long time to degrade in the body, making it difficult to control the quality standard of the product. At the same time, when used in food, probiotic microcapsules often have adverse effects on the sensory properties and flavor of food.

Some researchers embed probiotics in internal aqueous gelled multi-emulsion, but such emulsion is mainly designed for anti-digestion and intestinal colonization of probiotics, without considering long-term storage of probiotics. In addition, Cao Chen (China Oils and Fats 2019, 44 (12), 143-148), and Guo Zhanyang and Zheng Zhaojun (China Oils and Fats 2019, 44 (08), 65-71) studied the preparation and properties of gel emulsion, and found that the freeze-thaw process has a great influence on it. The freeze-thaw stability of the emulsion is poor, which means when the emulsion is applied in food, the freeze-thaw process will not only affect the stability of the emulsion, but also affect the activity of probiotics.

### SUMMARY OF THE PRESENT INVENTION

To overcome the defects in the prior art, the present invention provides a probiotic preparation based on a W₁/O/W₂ double emulsion structure, and a preparation method therefor and a use thereof, so as to solve some problems in the prior art or at least alleviate them.

The present invention provides a novel emulsion system containing probiotics, which can be applied to food, feed, animal health products, personal care or pharmaceutical products. The food, feed, animal health products, personal care or pharmaceutical products comprises the above emulsion, while the applications of the emulsion comprise, but are not limited to, gelato, ice cream, yogurt, cheese, beverages, animal health products, personal care or pharmaceutical products. The present invention solves the problem that the probiotics in the system can be easily inactivated during storage and freeze-thaw process, which improves the storage and freeze-thaw stability of the probiotics, and helps the probiotics to better exert their physiological activity.

Accordingly, the present invention provides a method for preparing a probiotic preparation based on a W₁/O/W₂ double emulsion structure, comprising steps of:
S1: processing probiotics or a mixture of probiotics and probiotic protective agents with a vortex treatment, so as to evenly disperse and form an internal water phase W₁;
S2: preliminarily mixing a lipid phase O with the internal water phase W₁, and stabilizing with emulsifiers; then preparing a water-in-oil primary emulsion Wi/O by stirring and low-energy emulsification or high-energy emulsification, thereby stabilizing the W₁ is in the lipid phase O; and
S3: using an emulsifier-contained solution as an external water phase W₂, and adding the external water phase W₂ to the primary emulsion W₁/O; then preparing a W₁/O/W₂ double emulsion by stirring, the methods of low-energy emulsification or the high-energy emulsification, thereby obtaining the probiotic preparation.

Preferably, in the step S1, the internal water phase W₁ contains the probiotics or the mixture of the probiotics and the probiotic protective agent;
the probiotic can be any probiotic, comprising Lactobacillus plantarum, Lactobacillus acidophilus, Lactobacillus rhamnosus, Lactobacillus bulgaricus, Bifidobacterium bifidum, Bifidobacterium longum, Lactococcus lactis and Streptococcus thermophilus;
the probiotic protective agent is selected from a group consisting of carbohydrates, proteins, amino acid salts, alcohols, inorganic salts, antioxidants, alkaloids, polymers and complexes; a concentration of the probiotic protective agent in the internal water phase is more than 0.01 wt%;
the probiotic protective agent in the step S1 is selected from a group consisting of glycerin, skimmed milk powder, ascorbic acid, whey protein isolate, trehalose, sucrose, fructose, maltose, lactose, dextran, gelatin, peptone, methylcellulose, betaine, sodium glutamate, xylitol, polyethylene glycol 1000 and sodium dodecyl sulfonate.

Preferably, in the step S2, the methods of low-energy emulsification contain phase transition, spontaneous emulsification, or membrane emulsification; the methods of high-energy emulsification contain high-speed shear, high-pressure homogeneous emulsification or microfluidic emulsification.

Preferably, the emulsifier in the step S2 is at least one of an oil-soluble emulsifier (at least one of natural or synthetic oil-soluble small molecule emulsifiers, oil-soluble macromolecular emulsifiers, oil-soluble polymers and complexes), a hydrophilic emulsifier (at least one of natural or synthetic hydrophilic small-molecule emulsifiers, hydrophilic macromolecular emulsifiers, hydrophilic polymers and complexes), and an amphiphilic emulsifier (at least one of natural or synthetic amphiphilic small molecule emulsifiers, amphiphilic macromolecular emulsifiers, amphiphilic polymers and complexes);

the oil-soluble emulsifier in the step S2 is selected form a group consisting of polyglycerol polyricinoleate (PGPR), Span 20, Span 60, Span 65, Span 80, Span 85, ethylene glycol fatty acid ester, propylene glycol monostearate ester, glyceryl monostearate, zein; the hydrophilic emulsifier is selected form a group consisting of Tween 80, pectin, hydroxypropyl methylcellulose, whey protein isolate, whey protein concentrate, casein, and β-lactoglobulin fiber; the amphiphilic emulsifier is selected form a group consisting of lecithin and sucrose ester.

Preferably, an adding amount of the emulsifier is at least 0.1 wt% of the lipid phase O.

Preferably, the lipid phase in the step S2 is selected from a group consisting of fat, sterol, and lipoid which are liquid at ambient temperature (20° C.), and from a group consisting of fat, sterol, and lipoid that are solid at ambient temperature;

the liquid fat is modified or unmodified vegetable and animal oils, which is selected from a group consisting of palm oil, avocado oil, mustard oil, linseed oil, grape oil, peanut oil, coconut oil, olive oil, thistle oil, grapeseed oil, sesame oil, soybean oil, sunflower oil, flaxseed oil, cotton oil, rapeseed oil, low-erucic acid rapeseed oil, corn oil, rice oil, safflower oil, kapok oil, sesame oil, evening primrose oil, fish oil, and seafood oil; the solid fat is modified or unmodified vegetable and animal oils, which is selected from a group consisting of cocoa butter, shea butter, sal resin, chicken fat, tallow, milk fat, and lard; the sterol and the lipid are selected from a group consisting of modified or unmodified beeswax, paraffin wax, sunflower wax, rice wax, candelilla wax, palm wax and sitosterol; modification refers to hydrogenation, fractionation and/or transesterification.

Preferably, the lipid phase is at most 90 wt% of a total emulsion weight.

Preferably, the low-energy emulsification is phase transition, spontaneous emulsification, or membrane emulsification; and the high-energy emulsification is high-speed shear, high-pressure homogeneous emulsification, or microfluidic emulsification.

Preferably, the emulsifier is selected from a group consisting of small molecule emulsifiers, polysaccharide emulsifiers, polypeptide emulsifiers, protein emulsifiers, polymers and complexes;

the emulsifier is selected from a group consisting of Tween 80, pectin, hydroxypropyl methylcellulose, whey protein isolate, whey protein concentrate, casein, and β-lactoglobulin fiber.

Preferably, in the external water phase W₂, a mass percentage of the emulsifier is at least 0.1 wt%.

Preferably, in the external water phase W₂, a volume ratio of the external water phase W₂ to the primary emulsion W₁/O is at most 99%;

A probiotic preparation based on the W₁/O/W₂ double emulsion structure is also provided, which is prepared by using the above-mentioned preparation method.

An application of the above probiotic preparation based on the W₁/O/W₂ double emulsion structure in probiotic products is also provided.

Preferably, the probiotic products comprise food (normal temperature or low temperature food), feed, animal health products, personal care or pharmaceutical products.

Preferably, the products comprise gelato, ice cream, yogurt, cheese, beverages, feed, animal health products, personal care or pharmaceutical products.

To sum up, advantages and positive effects of the present invention are as follows:

The applicants have been devoted to the research on the processing of probiotic preparations for a long time. After a lot of research and exploration, the above method for preparing probiotic preparations has been developed. The probiotic preparation prepared by the method can ensure the survival rate of the probiotics and prolong its storage time, thereby improving the stability of the probiotics, and preparing the probiotic preparation in which the probiotics show better physiological activity.

The present invention adopts the W₁/O/W₂ double emulsion system to protect and embed the probiotics. The probiotics embedding system can keep the probiotics active during storage and freezing and thawing, so as to reduce the external influence on the probiotics, and improve storage and freeze-thaw stability of the probiotics during the shelf life. At the same time, the probiotics are embedded in the internal water phase of the double emulsion, wherein oil film and interface film can protect the probiotics from the external environment such as oxygen, thereby further improving the survival rate of the probiotics, as well as the storage and freeze-thaw stability. The present invention effectively solves the problems of unstable quality of probiotic products, insufficient number of viable probiotics, and short survival time of probiotics in probiotic products.

The preparation process of the present invention is simple, so the cost is effectively reduced. The present invention has important application value for prolonging the probiotic survival rate during the shelf life of probiotic products, improving storage and freeze-thaw stability, and ensuring the active function of the probiotic products. The probiotic preparation based on the double emulsion can be used in gelato, ice cream, yogurt, cheese, beverages, feed, animal health products, personal care or pharmaceutical products, and has broad application prospects.

### BRIEF DESCRIPTION OF THE DRAWINGS

FIG. 1 illustrates a double emulsion sample with embedded probiotics;

FIG. 2 is a CLSM diagram of a double emulsion with the embedded probiotics;

FIG. 3 illustrates plate count detection results of an embodiment 1;

FIG. 4 illustrates plate count detection results of an embodiment 2;

FIG. 5 illustrates plate count detection results of an embodiment 3;

FIG. 6 illustrates plate count detection results of an embodiment 4;

FIG. 7 illustrates plate count detection results of an embodiment 5;

FIG. 8 illustrates plate count detection results of an embodiment 6;

FIG. 9 illustrates plate count detection results of an embodiment 7;

FIG. 10 illustrates plate count detection results of an embodiment 8;

FIG. 11 illustrates plate count detection results of an embodiment 9;

FIG. 12 illustrates plate count detection results of an embodiment 10;

FIG. 13 illustrates plate count detection results of an embodiment 11;

FIG. 14 illustrates plate count detection results of an embodiment 12;

FIG. 15 illustrates plate count detection results of an embodiment 13;

FIG. 16 illustrates plate count detection results of an embodiment 14;

FIG. 17 illustrates plate count detection results of an embodiment 15;

FIG. 18 illustrates plate count detection results of an embodiment 16;

FIG. 19 illustrates plate count detection results of an embodiment 17;

FIG. 20 illustrates plate count detection results of an embodiment 18; and

FIG. 21 illustrates plate count detection results of an embodiment 19.

### DETAILED DESCRIPTION OF THE PREFERRED EMBODIMENT

In order to make the object, technical solution and advantages clearer, the present invention will be further illustrated below in conjunction with preferred embodiments. The preferred embodiments described are exemplary only and not intended to be limiting.

The present invention provides a probiotic preparation based on a W₁/O/W₂ double emulsion structure, and a preparation method therefor and a use thereof, which will be described below. According to the present invention, the probiotic can be any probiotic, comprising Lactobacillus plantarum, Lactobacillus acidophilus, Lactobacillus rhamnosus, Lactobacillus bulgaricus, Bifidobacterium bifidum, Bifidobacterium longum, Lactococcus lactis, and Streptococcus thermophilus. Since there is no significant difference when applying different probiotics to the technical solution of the present invention, only some of the probiotics are described below as examples. The present invention has no special limitation on the lipid phase, as long as it can meet the relevant requirements for eating. Unless otherwise specified, the reagents, methods and equipment used in the present invention are common in the art, and the glassware, centrifuge tubes, pipette tips, and suspension and solution used in each embodiment are all sterilize at 121° C. for 15 min.

### Embodiment 1: Preparation of Double Emulsion Probiotic Preparation and Performance Test

1. A preparation method comprises steps of:

preparing probiotic suspension: activating the probiotic Lactobacillus plantarum with an MRS broth medium, culturing in a constant temperature incubator at 30° C. for 14 hours, and then centrifuging probiotic liquid to obtain the probiotic;

preparing the internal water phase W₁: preparing 10 wt% protective solution (trehalose here) as the probiotic protective agent, sterilizing at 121° C. for 15 min, and cooling to room temperature; mixing the probiotic with the probiotic protective agent, and dispersing evenly through vortex to form the internal water phase W₁;
wherein a concentration of the probiotic protective agent in the internal water phase is preferably at least 0.01 wt%, and more preferably 2-20 wt%;
wherein the probiotic protective agent is selected from a group consisting of carbohydrates, proteins, amino acid salts, alcohols, inorganic salts, antioxidants, alkaloids, polymers and complexes; preferably, the probiotic protective agent is selected from a group consisting of glycerin, skimmed milk powder, ascorbic acid, whey protein isolate, trehalose, sucrose, fructose, maltose, lactose, dextran, gelatin, peptone, methylcellulose, betaine, sodium glutamate, xylitol, polyethylene glycol 1000 and sodium dodecyl sulfonate;

preparing the lipid phase O: adding 6 g of emulsifier (polyglycerol polyricinoleate here) to 94 g of oil (soybean oil here), and stirring magnetically at 500 rpm for 5-45 min to obtain 6 wt% polyglycerol polyricinoleate , namely the lipid phase O;
wherein an adding amount of the emulsifier is preferably at least 0.1 wt% of the lipid phase O, more preferably 3%-10%, optimally 6%; magnetically stirring parameters are: 200-800 rpm for 5-45 min;
wherein the emulsifier is selected form a group consisting of an oil-soluble emulsifier, a hydrophilic emulsifier, and an amphiphilic emulsifier, such as polyglycerol polyricinoleate (PGPR), Span20, Span60, Span65, Span80, Span85, ethylene glycol fatty acid ester, propylene glycol monostearate ester, glyceryl monostearate, zein, Tween 80, pectin, hydroxypropyl methylcellulose, whey protein isolate, whey protein concentrate, casein, β-lactoglobulin fiber, lecithin and sucrose ester;

preparing the primary emulsion W₁/O: dropping the internal water phase W₁ into the lipid phase O while stirring, wherein a mass ratio of the internal water phase W₁ to the lipid phase O is 1:3, a stirring speed is 1000 rpm, and a dropping rate is 10-100 mL/min; then stirring for 2-5 min, and emulsifying by the high-energy emulsification (high-speed shear at 10000-18000 rpm for 8-15 min) to obtain the primary emulsion W₁/O;

wherein a mass ratio of the lipid phase O to the internal water phase W₁ is preferably 1-5:1, and more preferably 1-3:1; a stirring speed is 500-2000 rpm, preferably 500-1500 rpm;

preparing the external water phase W₂: adding 3 g of hydrophilic emulsifier (whey protein isolate here) to 97 g of deionized water, stirring for 2 hours to fully dissolve, and then putting in a 4° C. refrigerator overnight to fully hydrate, so as to obtain 3 wt% whey protein isolate solution, which is the external water phase W₂;

wherein the hydrophilic emulsifier is selected from a group consisting of small molecule emulsifiers, polysaccharide emulsifiers, polypeptide emulsifiers, protein emulsifiers, polymers and complexes; preferably from Tween 80, pectin, hydroxypropyl methylcellulose, whey protein isolate, whey protein concentrate, casein, and β-lactoglobulin fiber; in the external water phase W₂, a mass percentage of the emulsifier is preferably at least 0.1 wt%, and more preferably 1-10 wt%; and

preparing the W₁/O/W₂ double emulsion: mixing the primary emulsion W₁/O and the external water phase W₂ at a mass ratio of 1:2, and emulsifying by the high-energy emulsification (high-speed shear at 10000-18000 rpm for 2-5 min) to obtain the W₁/O/W₂ double emulsion; storing at a low temperature of -20 to 4° C. to obtain the probiotic preparation;

wherein a volume ratio of the external water phase W₂ to the primary emulsion W₁/O is at most 99%, preferably 1-50: 1, more preferably 1-20: 1, optimally 2-4:1.

2. Performance test:

using original probiotic solution and Lactobacillus plantarum suspended in sterile water as W₁, and preparing W₁/O/W₂ double emulsion respectively by low-speed stirring and stirring with shearing as a control group; using Lactobacillus plantarum suspended in 10 wt% trehalose solution as W₁, and preparing W₁/O/W₂ double emulsion only by stirring as a control group; using the Lactobacillus plantarum embedded in the double emulsion W₁/O/W₂ of the embodiment 1 as an experimental group, so as to observe the effects of emulsion preparation conditions and protective agent on Lactobacillus plantarum viable count in the W₁/O/W₂ double emulsion; collecting equal amounts of appropriate samples before the control group and the experimental group are frozen at -18° C. for 12 hours, and after stored at 4° C. until completely thawed; determining the viable count by the dilution spread plate method.

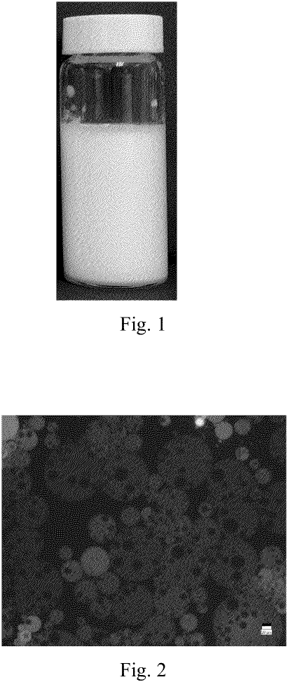

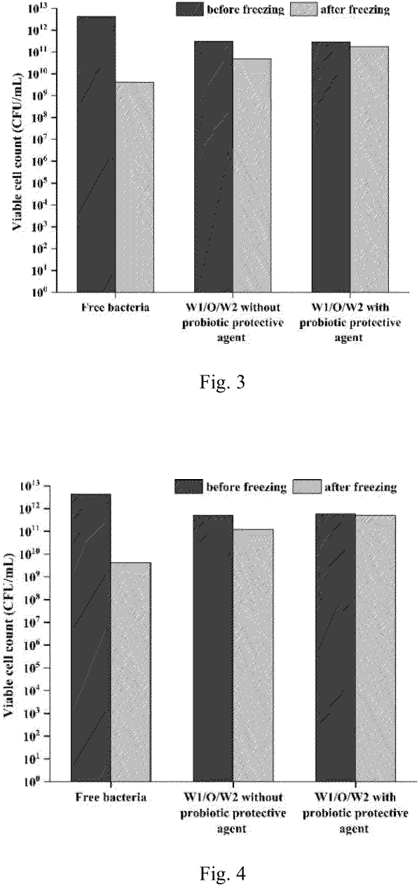

The emulsion prepared by the present invention is shown in FIG. 1, and CLSM diagram is shown in FIG. 2 (the gray part is the oil phase, which is dyed with Nile Red dye, and an excitation wavelength thereof is 483 nm; the black part is undyed water phase). Analysis results are shown in FIG. 3, which show that for Lactobacillus plantarum before freezing and thawing, the viable count in the emulsion prepared by stirring is reasonably reduced, while the viable count in the emulsion prepared by shearing is approximately equal to or higher than that in the emulsion prepared by stirring. Therefore, it is considered that shearing only makes the emulsion droplets smaller, and in the same amount of emulsion, more droplets are spread on the solid medium, providing more viable probiotics. This further illustrates that the preparation conditions of the present invention have no impact on the survival rate of probiotics.

After freezing and thawing, the viable count of Lactobacillus plantarum decreased significantly: viable count in the original probiotic solution before and after freezing and thawing is 4.30×10¹² cfu/mL and 4.26× 10⁹ cfu/mL, and the survival rate is 0.10%. For the emulsion prepared by shearing, viable count in a sample without protective agent before and after freezing and thawing is 3.05×10¹¹ cfu/mL and 5.10×10¹⁰ cfu/mL, and the survival rate is 16.72%, while viable count in a sample with the protective agent before and after freezing and thawing is 2.91×10¹¹cfu/mL and 1.79×10¹¹ cfu/mL, and the survival rate is 61.51%. These data indicate that the W₁/O/W₂ double emulsion structure and trehalose have a certain protective effect on Lactobacillus plantarum, especially on the product prepared in the embodiment 1 of the present invention, wherein the viable count after freezing and thawing is significantly higher than those of other groups, indicating that the probiotic preparation of the present invention can inhibit the inactivation of the probiotics, so as to better exert their physiological activity in subsequent applications.

### Embodiment 2: Preparation of Double Emulsion Probiotic Preparation

1. A preparation method comprises steps of:

(1) preparing probiotic suspension: activating the probiotic Lactobacillus plantarum with an MRS broth medium, culturing in a constant temperature incubator at 30° C. for 14 hours, and then centrifuging probiotic liquid to obtain the probiotic;
(2) preparing the internal water phase W₁: preparing 10 wt% protective solution (trehalose here) as the probiotic protective agent, sterilizing at 121° C. for 15 min, and cooling to room temperature; mixing the probiotic with the probiotic protective agent, and dispersing evenly through vortex to form the internal water phase W₁;
(3) preparing the emulsifier: mixing 3 g of polyglycerol polyricinoleate and 3 g of lecithin, and stirring magnetically at 500 rpm at 50° C. for 15-30 min, so as to obtain an emulsifier complex of an oil-soluble emulsifier and an amphiphilic emulsifier;
(4) preparing the lipid phase O: adding 6 g of emulsifier complex (the emulsifier complex of oil-soluble emulsifier polyglycerol polyricinoleate and amphiphilic emulsifier lecithin) to 94 g of oil (soybean oil here), and stirring magnetically at 500 rpm for 5-45 min to obtain 6 wt% emulsifier complex, namely the lipid phase O;
(5) preparing the primary emulsion W₁/O: dropping the internal water phase W₁ into the lipid phase O while stirring, wherein a mass ratio of the internal water phase W₁ to the lipid phase O is 1:3, a stirring speed is 1000 rpm, and a dropping rate is 10-100 mL/min; then stirring for 2-5 min, and emulsifying by the high-energy emulsification (high-speed shear at 10000-18000 rpm for 8-15 min) to obtain the primary emulsion W₁/O;
(6) preparing the external water phase W₂: adding 3 g of hydrophilic emulsifier (whey protein isolate here) to 97 g of deionized water, stirring for 2 hours to fully dissolve, and then putting in a 4° C. refrigerator overnight to fully hydrate, so as to obtain 3 wt% whey protein isolate solution, which is the external water phase W₂; and
(7) preparing the W₁/O/W₂ double emulsion: mixing the primary emulsion W₁/O and the external water phase W₂ at a mass ratio of 1:2, and emulsifying by the high-energy emulsification (high-speed shear at 10000-18000 rpm for 2-5 min) to obtain the W₁/O/W₂ double emulsion; storing at a low temperature of -20 to 4° C. to obtain the probiotic preparation.

2. Performance test:

using original probiotic solution and W₁/O/W₂ double emulsion without protective agent as a control group; using the Lactobacillus plantarum embedded in the double emulsion W₁/O/W₂ of the embodiment 2 as an experimental group, so as to observe the effects of protective agent on Lactobacillus plantarum viable count in the W₁/O/W₂ double emulsion; collecting equal amounts of appropriate samples before the control group and the experimental group are frozen at -18° C., after frozen at -18° C. for 12 hours, and after stored at 4° C. until completely thawed; determining the viable count by the dilution spread plate method.

Analysis results are shown in FIG. 4, it can be seen that the viable count of Lactobacillus plantarum decreases after freezing and thawing, and decrements vary for different samples. Viable count in the original probiotic solution before and after freezing and thawing is 4.30×10¹² cfu/mL and 4.26× 10⁹ cfu/mL, and the survival rate is 0.10%. Viable count in a product without protective agent before and after freezing and thawing is 5.05×10¹¹ cfu/mL and 1.20×10¹¹ cfu/mL, and the survival rate is 23.76%, while viable count in a product prepared in the embodiment 2 before and after freezing and thawing is 5.91×10¹¹ cfu/mL and 4.90×10¹¹ cfu/mL, and the survival rate is 82.91%. These data indicate that the W₁/O/W₂ double emulsion structure and the protective agent have a protective effect on Lactobacillus plantarum, indicating that the probiotic preparation of the present invention can inhibit the inactivation of the probiotics to a certain degree, so as to better exert their physiological activity in subsequent applications.

### Embodiment 3: Preparation of Double Emulsion Probiotic Preparation

1. A preparation method comprises steps of:

(1) preparing probiotic suspension: activating the probiotic Lactobacillus plantarum with an MRS broth medium, culturing in a constant temperature incubator at 30° C. for 14 hours, and then centrifuging probiotic liquid to obtain the probiotic;
(2) preparing the internal water phase W₁: preparing 10 wt% protective solution (trehalose here) as the probiotic protective agent, sterilizing at 121° C. for 15 min, and cooling to room temperature; mixing the probiotic with the probiotic protective agent, and dispersing evenly through vortex to form the internal water phase W₁;
(3) preparing the emulsifier: mixing 3 g of polyglycerol polyricinoleate and 3 g of Tween 80, and stirring magnetically at 500 rpm at 50° C. for 15-30 min, so as to obtain an emulsifier complex of an oil-soluble emulsifier and a hydrophilic emulsifier;
(4) preparing the lipid phase O: adding 6 g of emulsifier complex (the emulsifier complex of oil-soluble emulsifier polyglycerol polyricinoleate and hydrophilic emulsifier Tween 80) to 94 g of oil (soybean oil here), and stirring magnetically at 500 rpm for 5-45 min to obtain 6 wt% emulsifier complex, namely the lipid phase O;
(5) preparing the primary emulsion W₁/O: dropping the internal water phase W₁ into the lipid phase O while stirring, wherein a mass ratio of the internal water phase W₁ to the lipid phase O is 1:3, a stirring speed is 1000 rpm, and a dropping rate is 10-100 mL/min; then stirring for 2-5 min, and emulsifying by the high-energy emulsification (high-speed shear at 10000-18000 rpm for 8-15 min) to obtain the primary emulsion W₁/O;
(6) preparing the external water phase W₂: adding 3 g of hydrophilic emulsifier (whey protein isolate here) to 97 g of deionized water, stirring for 2 hours to fully dissolve, and then putting in a 4° C. refrigerator overnight to fully hydrate, so as to obtain 3 wt% whey protein isolate solution, which is the external water phase W₂; and
(7) preparing the W₁/O/W₂ double emulsion: mixing the primary emulsion W1/O and the external water phase W₂ at a mass ratio of 1:2, and emulsifying by the high-energy emulsification (high-speed shear at 10000-18000 rpm for 2-5 min) to obtain the W₁/O/W₂ double emulsion; storing at a low temperature of -20 to 4° C. to obtain the probiotic preparation.

2. Performance test:

using original probiotic solution and W₁/O/W₂ double emulsion without protective agent as a control group; using the Lactobacillus plantarum embedded in the double emulsion W₁/O/W₂ of the embodiment 3 as an experimental group, so as to observe the effects of protective agent on Lactobacillus plantarum viable count in the W₁/O/W₂ double emulsion; collecting equal amounts of appropriate samples before the control group and the experimental group are frozen at -18° C., after frozen at -18° C. for 12 hours, and after stored at 4° C. until completely thawed; determining the viable count by the dilution spread plate method.

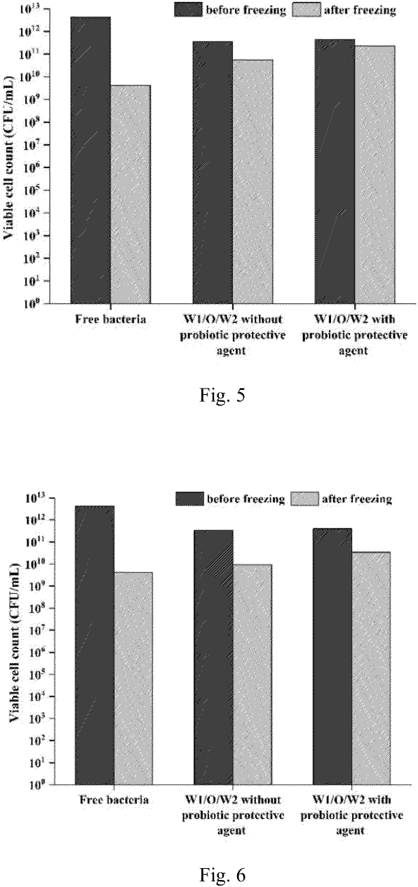

Analysis results are shown in FIG. 5, it can be seen that the viable count of Lactobacillus plantarum decreases after freezing and thawing, and decrements vary for different samples. Viable count in the original probiotic solution before and after freezing and thawing is 4.30×10¹² cfu/mL and 4.26× 10⁹ cfu/mL, and the survival rate is 0.10%. Viable count in a product without protective agent before and after freezing and thawing is 3.43×10¹¹ cfu/mL and 5.69×10¹⁰ cfu/mL, and the survival rate is 16.59%, while viable count in a product prepared in the embodiment 3 before and after freezing and thawing is 4.26× 10¹¹ cfu/mL and 2.33×10¹¹ cfu/mL, and the survival rate is 54.69%. These data indicate that the W₁/O/W₂ double emulsion structure and the protective agent have a protective effect on Lactobacillus plantarum, indicating that the probiotic preparation of the present invention can inhibit the inactivation of the probiotics to a certain degree, so as to better exert their physiological activity in subsequent applications.

### Embodiment 4: Preparation of Double Emulsion Probiotic Preparation

1. A preparation method comprises steps of:

(1) preparing probiotic suspension: activating the probiotic Lactobacillus plantarum with an MRS broth medium, culturing in a constant temperature incubator at 30° C. for 14 hours, and then centrifuging probiotic liquid to obtain the probiotic;
(2) preparing the internal water phase W₁: preparing 10 wt% protective solution (trehalose here) as the probiotic protective agent, sterilizing at 121° C. for 15 min, and cooling to room temperature; mixing the probiotic with the probiotic protective agent, and dispersing evenly through vortex to form the internal water phase W₁;
(3) preparing the emulsifier: mixing 3 g of lecithin and 3 g of Tween 80, and stirring magnetically at 500 rpm at 50° C. for 15-30 min, so as to obtain an emulsifier complex of an amphiphilic emulsifier and a hydrophilic emulsifier;
(4) preparing the lipid phase O: adding 6 g of emulsifier complex (the emulsifier complex of amphiphilic emulsifier lecithin and hydrophilic emulsifier Tween 80) to 94 g of oil (soybean oil here), and stirring magnetically at 500 rpm for 5-45 min to obtain 6 wt% emulsifier complex, namely the lipid phase O;
(5) preparing the primary emulsion W₁/O: dropping the internal water phase W₁ into the lipid phase O while stirring, wherein a mass ratio of the internal water phase W₁ to the lipid phase O is 1:3, a stirring speed is 1000 rpm, and a dropping rate is 10-100 mL/min; then stirring for 2-5 min, and emulsifying by the high-energy emulsification (high-speed shear at 10000-18000 rpm for 8-15 min) to obtain the primary emulsion W₁/O;
(6) preparing the external water phase W₂: adding 3 g of hydrophilic emulsifier (whey protein isolate here) to 97 g of deionized water, stirring for 2 hours to fully dissolve, and then putting in a 4° C. refrigerator overnight to fully hydrate, so as to obtain 3 wt% whey protein isolate solution, which is the external water phase W₂; and
(7) preparing the W₁/O/W₂ double emulsion: mixing the primary emulsion W1/O and the external water phase W₂ at a mass ratio of 1:2, and emulsifying by the high-energy emulsification (high-speed shear at 10000-18000 rpm for 2-5 min) to obtain the W₁/O/W₂ double emulsion; storing at a low temperature of -20 to 4° C. to obtain the probiotic preparation.

2. Performance test:

using original probiotic solution and W₁/O/W₂ double emulsion without protective agent as a control group; using the Lactobacillus plantarum embedded in the double emulsion W₁/O/W₂ of the embodiment 4 as an experimental group, so as to observe the effects of protective agent on Lactobacillus plantarum viable count in the W₁/O/W₂ double emulsion; collecting equal amounts of appropriate samples before the control group and the experimental group are frozen at -18° C. for 12 hours, and after stored at 4° C. until completely thawed; determining the viable count by the dilution spread plate method.

Analysis results are shown in FIG. 6, it can be seen that the viable count of Lactobacillus plantarum decreases after freezing and thawing, and decrements vary for different samples. Viable count in the original probiotic solution before and after freezing and thawing is 4.30×10¹² cfu/mL and 4.26× 10⁹ cfu/mL, and the survival rate is 0.10%. Viable count in a product without protective agent before and after freezing and thawing is 3.39×10¹¹ cfu/mL and 9.56×10⁹ cfu/mL, and the survival rate is 2.82%, while viable count in a product prepared in the embodiment 4 before and after freezing and thawing is 4.02×10¹¹ cfu/mL and 3.40×10¹⁰ cfu/mL, and the survival rate is 8.46%. These data indicate that the W₁/O/W₂ double emulsion structure and the protective agent have a protective effect on Lactobacillus plantarum, indicating that the probiotic preparation of the present invention can inhibit the inactivation of the probiotics to a certain degree, so as to better exert their physiological activity in subsequent applications.

### Embodiment 5: Preparation of Double Emulsion Probiotic Preparation

1. A preparation method comprises steps of:

(1) preparing probiotic suspension: activating the probiotic Lactobacillus plantarum with an MRS broth medium, culturing in a constant temperature incubator at 30° C. for 14 hours, and then centrifuging probiotic liquid to obtain the probiotic;
(2) preparing the internal water phase W₁: preparing 10 wt% protective solution (trehalose here) as the probiotic protective agent, sterilizing at 121° C. for 15 min, and cooling to room temperature; mixing the probiotic with the probiotic protective agent, and dispersing evenly through vortex to form the internal water phase W₁;
(3) preparing the lipid phase O: adding 6 g of emulsifier (hydrophilic emulsifier Tween 80 here) to 94 g of oil (soybean oil here), and stirring magnetically at 500 rpm for 5-45 min to obtain 6 wt% Tween 80, namely the lipid phase O;
(4) preparing the primary emulsion W₁/O: dropping the internal water phase W₁ into the lipid phase O while stirring, wherein a mass ratio of the internal water phase W₁ to the lipid phase O is 1:3, a stirring speed is 1000 rpm, and a dropping rate is 10-100 mL/min; then stirring for 2-5 min, and emulsifying by the high-energy emulsification (high-speed shear at 10000-18000 rpm for 8-15 min) to obtain the primary emulsion W₁/O;
(5) preparing the external water phase W₂: adding 3 g of hydrophilic emulsifier (whey protein isolate here) to 97 g of deionized water, stirring for 2 hours to fully dissolve, and then putting in a 4° C. refrigerator overnight to fully hydrate, so as to obtain 3 wt% whey protein isolate solution, which is the external water phase W₂; and
(6) preparing the W₁/O/W₂ double emulsion: mixing the primary emulsion W1/O and the external water phase W₂ at a mass ratio of 1:2, and emulsifying by the high-energy emulsification (high-speed shear at 10000-18000 rpm for 2-5 min) to obtain the W₁/O/W₂ double emulsion; storing at a low temperature of -20 to 4° C. to obtain the probiotic preparation.

2. Performance test:

using original probiotic solution and W₁/O/W₂ double emulsion without protective agent as a control group; using the Lactobacillus plantarum embedded in the double emulsion W₁/O/W₂ of the embodiment 5 as an experimental group, so as to observe the effects of protective agent on Lactobacillus plantarum viable count in the W₁/O/W₂ double emulsion; collecting equal amounts of appropriate samples before the control group and the experimental group are frozen at -18° C., after frozen at -18° C. for 12 hours, and after stored at 4° C. until completely thawed; determining the viable count by the dilution spread plate method.

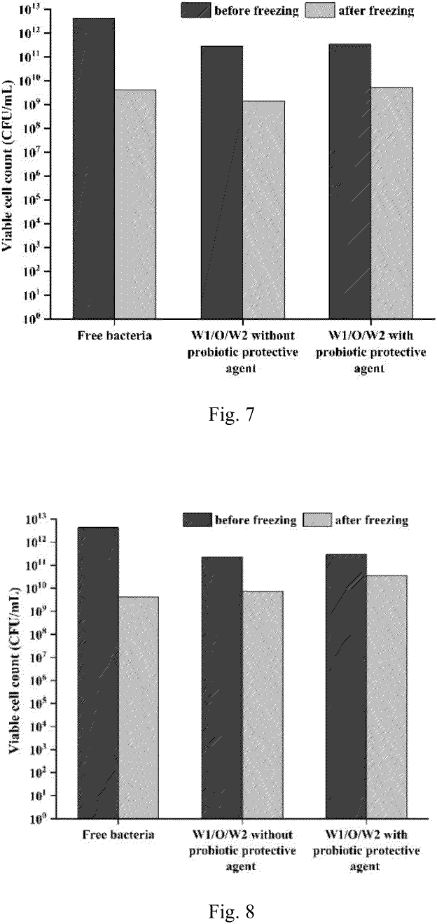

Analysis results are shown in FIG. 7, it can be seen that the viable count of Lactobacillus plantarum decreases after freezing and thawing. Viable count in the original probiotic solution before and after freezing and thawing is 4.30×10¹² cfu/mL and 4.26× 10⁹ cfu/mL, and the survival rate is 0.10%. Viable count in a product without protective agent before and after freezing and thawing is 2.89×10¹¹ cfu/mL and 1.42×10⁹ cfu/mL, and the survival rate is 0.49%, while viable count in a product prepared in the embodiment 5 before and after freezing and thawing is 3.45×10¹¹ cfu/mL and 5.21×10⁹ cfu/mL, and the survival rate is 1.51%. These data indicate that the product prepared by the embodiment 5 provides relatively weak protection to Lactobacillus plantarum.

### Embodiment 6: Preparation of Double Emulsion Probiotic Preparation

1. A preparation method comprises steps of:

(1) preparing probiotic suspension: activating the probiotic Lactobacillus plantarum with an MRS broth medium, culturing in a constant temperature incubator at 30° C. for 14 hours, and then centrifuging probiotic liquid to obtain the probiotic;
(2) preparing the internal water phase W₁: preparing 10 wt% protective solution (trehalose here) as the probiotic protective agent, sterilizing at 121° C. for 15 min, and cooling to room temperature; mixing the probiotic with the probiotic protective agent, and dispersing evenly through vortex to form the internal water phase W₁;
(3) preparing the lipid phase O: adding 6 g of emulsifier (amphiphilic emulsifier lecithin here) to 94 g of oil (soybean oil here), and stirring magnetically at 500 rpm for 5-45 min to obtain 6 wt% lecithin, namely the lipid phase O;
(4) preparing the primary emulsion W₁/O: dropping the internal water phase W₁ into the lipid phase O while stirring, wherein a mass ratio of the internal water phase W₁ to the lipid phase O is 1:3, a stirring speed is 1000 rpm, and a dropping rate is 10-100 mL/min; then stirring for 2-5 min, and emulsifying by the high-energy emulsification (high-speed shear at 10000-18000 rpm for 8-15 min) to obtain the primary emulsion W₁/O;
(5) preparing the external water phase W₂: adding 3 g of hydrophilic emulsifier (whey protein isolate here) to 97 g of deionized water, stirring for 2 hours to fully dissolve, and then putting in a 4° C. refrigerator overnight to fully hydrate, so as to obtain 3 wt% whey protein isolate solution, which is the external water phase W₂; and
(6) preparing the W₁/O/W₂ double emulsion: mixing the primary emulsion Wi/O and the external water phase W₂ at a mass ratio of 1:2, and emulsifying by the high-energy emulsification (high-speed shear at 10000-18000 rpm for 2-5 min) to obtain the W₁/O/W₂ double emulsion; storing at a low temperature of -20 to 4° C. to obtain the probiotic preparation.

2. Performance test:

using original probiotic solution and W₁/O/W₂ double emulsion without protective agent as a control group; using the Lactobacillus plantarum embedded in the double emulsion W₁/O/W₂ of the embodiment 6 as an experimental group, so as to observe the effects of protective agent on Lactobacillus plantarum viable count in the W₁/O/W₂ double emulsion; collecting equal amounts of appropriate samples before the control group and the experimental group are frozen at -18° C. for 12 hours, and after stored at 4° C. until completely thawed; determining the viable count by the dilution spread plate method.

Analysis results are shown in FIG. 8, it can be seen that the viable count of Lactobacillus plantarum decreases after freezing and thawing. Viable count in the original probiotic solution before and after freezing and thawing is 4.30×10¹² cfu/mL and 4.26× 10⁹ cfu/mL, and the survival rate is 0.10%. Viable count in a product without protective agent before and after freezing and thawing is 224×10¹¹ cfu/mL and 7.59×10⁹ cfu/mL, and the survival rate is 3.39%, while viable count in a product prepared in the embodiment 6 before and after freezing and thawing is 2.96×10¹¹ cfu/mL and 3.58×10¹⁰ cfu/mL, and the survival rate is 12.09%. These data indicate that the product prepared by the embodiment 6 provides relatively weak protection to Lactobacillus plantarum.

### Embodiment 7: Preparation of Double Emulsion Probiotic Preparation

1. A preparation method comprises steps of:

(1) preparing probiotic suspension: activating the probiotic Lactobacillus plantarum with an MRS broth medium, culturing in a constant temperature incubator at 30° C. for 14 hours, and then centrifuging probiotic liquid to obtain the probiotic;
(2) preparing the internal water phase W₁: preparing 0.1 wt% protective solution (trehalose here) as the probiotic protective agent, sterilizing at 121° C. for 15 min, and cooling to room temperature; mixing the probiotic with the probiotic protective agent, and dispersing evenly through vortex to form the internal water phase W₁;
(3) preparing the lipid phase O: adding 6 g of emulsifier (polyglycerol polyricinoleate here) to 94 g of oil (soybean oil here), and stirring magnetically at 500 rpm for 5-45 min to obtain 6 wt% polyglycerol polyricinoleate, namely the lipid phase O;
(4) preparing the primary emulsion W₁/O: dropping the internal water phase W₁ into the lipid phase O while stirring, wherein a mass ratio of the internal water phase W₁ to the lipid phase O is 1:3, a stirring speed is 1000 rpm, and a dropping rate is 10-100 mL/min; then stirring for 2-5 min, and emulsifying by the high-energy emulsification (high-speed shear at 10000-18000 rpm for 8-15 min) to obtain the primary emulsion W₁/O;
(5) preparing the external water phase W₂: adding 3 g of hydrophilic emulsifier (whey protein isolate here) to 97 g of deionized water, stirring for 2 hours to fully dissolve, and then putting in a 4° C. refrigerator overnight to fully hydrate, so as to obtain 3 wt% whey protein isolate solution, which is the external water phase W₂; and
(6) preparing the W₁/O/W₂ double emulsion: mixing the primary emulsion W₁/O and the external water phase W₂ at a mass ratio of 1:2, and emulsifying by the high-energy emulsification (high-speed shear at 10000-18000 rpm for 2-5 min) to obtain the W₁/O/W₂ double emulsion; storing at a low temperature of -20 to 4° C. to obtain the probiotic preparation.

2. Performance test:

using original probiotic solution and W₁/O/W₂ double emulsion without protective agent as a control group; using the Lactobacillus plantarum embedded in the double emulsion W₁/O/W₂ of the embodiment 7 as an experimental group, so as to observe the effects of protective agent on Lactobacillus plantarum viable count in the W₁/O/W₂ double emulsion; collecting equal amounts of appropriate samples before the control group and the experimental group are frozen at -18° C., after frozen at -18° C. for 12 hours, and after stored at 4° C. until completely thawed; determining the viable count by the dilution spread plate method.

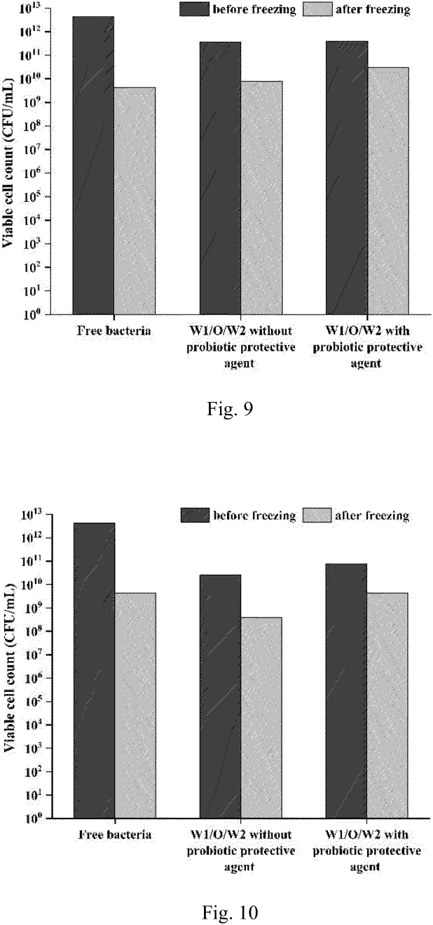

Analysis results are shown in FIG. 9, it can be seen that the viable count of Lactobacillus plantarum decreases after freezing and thawing, and decrements vary for different samples. Viable count in the original probiotic solution before and after freezing and thawing is 4.30×10¹² cfu/mL and 4.26× 10⁹ cfu/mL, and the survival rate is 0.10%. Viable count in a product without protective agent before and after freezing and thawing is 3.65×10¹¹ cfu/mL and 7.99×10⁹ cfu/mL, and the survival rate is 2.19%, while viable count in a product prepared in the embodiment 7 before and after freezing and thawing is 3.84×10¹¹ cfu/mL and 3.07×10¹⁰ cfu/mL, and the survival rate is 7.99%. These data indicate that the W₁/O/W₂ double emulsion structure and the protective agent have a protective effect on Lactobacillus plantarum, indicating that the probiotic preparation of the present invention can inhibit the inactivation of the probiotics to a certain degree, so as to better exert their physiological activity in subsequent applications.

### Embodiment 8: Preparation of Double Emulsion Probiotic Preparation

1. A preparation method comprises steps of:

(1) preparing probiotic suspension: activating the probiotic Lactobacillus plantarum with an MRS broth medium, culturing in a constant temperature incubator at 30° C. for 14 hours, and then centrifuging probiotic liquid to obtain the probiotic;
(2) preparing the internal water phase W₁: preparing 10 wt% protective solution (trehalose here) as the probiotic protective agent, sterilizing at 121° C. for 15 min, and cooling to room temperature; mixing the probiotic with the probiotic protective agent, and dispersing evenly through vortex to form the internal water phase W₁;
(3) preparing the lipid phase O: adding 0.1 g of emulsifier (polyglycerol polyricinoleate here) to 99.9 g of oil (soybean oil here), and stirring magnetically at 500 rpm for 5-45 min to obtain 0.1 wt% polyglycerol polyricinoleate, namely the lipid phase O;
(4) preparing the primary emulsion W₁/O: dropping the internal water phase W₁ into the lipid phase O while stirring, wherein a mass ratio of the internal water phase W₁ to the lipid phase O is 1:3, a stirring speed is 1000 rpm, and a dropping rate is 10-100 mL/min; then stirring for 2-5 min, and emulsifying by the high-energy emulsification (high-speed shear at 10000-18000 rpm for 8-15 min) to obtain the primary emulsion W₁/O;
(5) preparing the external water phase W₂: adding 3 g of hydrophilic emulsifier (whey protein isolate here) to 97 g of deionized water, stirring for 2 hours to fully dissolve, and then putting in a 4° C. refrigerator overnight to fully hydrate, so as to obtain 3 wt% whey protein isolate solution, which is the external water phase W₂; and
(6) preparing the W₁/O/W₂ double emulsion: mixing the primary emulsion W₁/O and the external water phase W₂ at a mass ratio of 1:2, and emulsifying by the high-energy emulsification (high-speed shear at 10000-18000 rpm for 2-5 min) to obtain the W₁/O/W₂ double emulsion; storing at a low temperature of -20 to 4° C. to obtain the probiotic preparation.

2. Performance test:

using original probiotic solution and W₁/O/W₂ double emulsion without protective agent as a control group; using the Lactobacillus plantarum embedded in the double emulsion W₁/O/W₂ of the embodiment 8 as an experimental group, so as to observe the effects of protective agent on Lactobacillus plantarum viable count in the W₁/O/W₂ double emulsion; collecting equal amounts of appropriate samples before the control group and the experimental group are frozen at -18° C., after frozen at -18° C. for 12 hours, and after stored at 4° C. until completely thawed; determining the viable count by the dilution spread plate method.

Analysis results are shown in FIG. 10, it can be seen that the viable count of Lactobacillus plantarum decreases after freezing and thawing, and decrements vary for different samples. Viable count in the original probiotic solution before and after freezing and thawing is 4.30×10¹² cfu/mL and 4.26× 10⁹ cfu/mL, and the survival rate is 0.10%. Viable count in a product without protective agent before and after freezing and thawing is 2.49×10¹⁰ cfu/mL and 3.83×10⁸ cfu/mL, and the survival rate is 1.54%, while viable count in a product prepared in the embodiment 8 before and after freezing and thawing is 7.87×10¹⁰ cfu/mL and 4.41×10⁹ cfu/mL, and the survival rate is 5.60%. These data indicate that the W₁/O/W₂ double emulsion structure and the protective agent have a protective effect on Lactobacillus plantarum, indicating that the probiotic preparation of the present invention can inhibit the inactivation of the probiotics to a certain degree, so as to better exert their physiological activity in subsequent applications.

### Embodiment 9: Preparation of Double Emulsion Probiotic Preparation

1. A preparation method comprises steps of:

(1) preparing probiotic suspension: activating the probiotic Lactobacillus plantarum with an MRS broth medium, culturing in a constant temperature incubator at 30° C. for 14 hours, and then centrifuging probiotic liquid to obtain the probiotic;
(2) preparing the internal water phase W₁: preparing 10 wt% protective solution (trehalose here) as the probiotic protective agent, sterilizing at 121° C. for 15 min, and cooling to room temperature; mixing the probiotic with the probiotic protective agent, and dispersing evenly through vortex to form the internal water phase W₁;
(3) preparing the lipid phase O: adding 6 g of emulsifier (polyglycerol polyricinoleate here) to 94 g of oil (soybean oil here), and stirring magnetically at 500 rpm for 5-45 min to obtain 6 wt% polyglycerol polyricinoleate, namely the lipid phase O;
(4) preparing the primary emulsion W₁/O: dropping the internal water phase W₁ into the lipid phase O while stirring, wherein a mass ratio of the internal water phase W₁ to the lipid phase O is 1:3, a stirring speed is 1000 rpm, and a dropping rate is 10-100 mL/min; then stirring for 2-5 min, and emulsifying by the high-energy emulsification (high-speed shear at 10000-18000 rpm for 8-15 min) to obtain the primary emulsion W₁/O;
(5) preparing the external water phase W₂: adding 3 g of hydrophilic emulsifier (β-lactoglobulin fiber here) to 97 g of deionized water, stirring for 2 hours to fully dissolve, and then putting in a 4° C. refrigerator overnight to fully hydrate, so as to obtain 3 wt% β-lactoglobulin fiber solution, which is the external water phase W₂; and
(6) preparing the W₁/O/W₂ double emulsion: mixing the primary emulsion Wi/O and the external water phase W₂ at a mass ratio of 1:2, and emulsifying by the high-energy emulsification (high-speed shear at 10000-18000 rpm for 2-5 min) to obtain the W₁/O/W₂ double emulsion; storing at a low temperature of -20 to 4° C. to obtain the probiotic preparation.

2. Performance test:

using original probiotic solution and W₁/O/W₂ double emulsion without protective agent as a control group; using the Lactobacillus plantarum embedded in the double emulsion W₁/O/W₂ of the embodiment 9 as an experimental group, so as to observe the effects of protective agent on Lactobacillus plantarum viable count in the W₁/O/W₂ double emulsion; collecting equal amounts of appropriate samples before the control group and the experimental group are frozen at -18° C., after frozen at -18° C. for 12 hours, and after stored at 4° C. until completely thawed; determining the viable count by the dilution spread plate method.

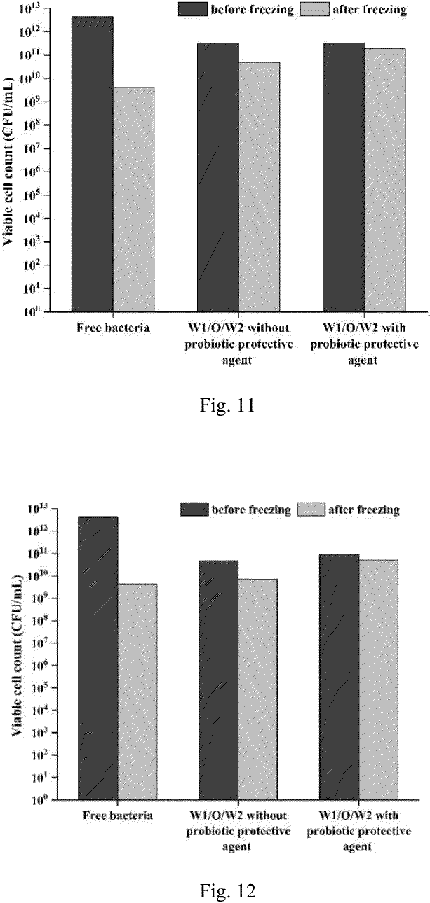

Analysis results are shown in FIG. 11, it can be seen that the viable count of Lactobacillus plantarum decreases after freezing and thawing, and decrements vary for different samples. Viable count in the original probiotic solution before and after freezing and thawing is 4.30×10¹² cfu/mL and 4.26× 10⁹ cfu/mL, and the survival rate is 0.10%. Viable count in the product of double emulsion without protective agent before and after freezing and thawing is 3.11×10¹¹ cfu/mL and 4.85×10¹⁰ cfu/mL, and the survival rate is 15.59%, while viable count in a product prepared in the embodiment 9 before and after freezing and thawing is 3.28×10¹¹ cfu/mL and 1.89×10¹¹ cfu/mL, and the survival rate is 57.62%. These data indicate that the W₁/O/W₂ double emulsion structure and the protective agent have a protective effect on Lactobacillus plantarum, indicating that the probiotic preparation of the present invention can inhibit the inactivation of the probiotics, so as to better exert their physiological activity in subsequent applications.

### Embodiment 10: Preparation of Double Emulsion Probiotic Preparation

1. A preparation method comprises steps of:

(1) preparing probiotic suspension: activating the probiotic Lactobacillus plantarum with an MRS broth medium, culturing in a constant temperature incubator at 30° C. for 14 hours, and then centrifuging probiotic liquid to obtain the probiotic;
(2) preparing the internal water phase W₁: preparing 10 wt% protective solution (trehalose here) as the probiotic protective agent, sterilizing at 121° C. for 15 min, and cooling to room temperature; mixing the probiotic with the probiotic protective agent, and dispersing evenly through vortex to form the internal water phase W₁;
(3) preparing the lipid phase O: adding 6 g of emulsifier (polyglycerol polyricinoleate here) to 94 g of oil (soybean oil here), and stirring magnetically at 500 rpm for 5-45 min to obtain 6 wt% polyglycerol polyricinoleate, namely the lipid phase O;
(4) preparing the primary emulsion W₁/O: dropping 5 g of the internal water phase W₁ into 100 g of the lipid phase O while stirring, wherein a stirring speed is 1000 rpm, and a dropping rate is 10-100 mL/min; then stirring for 2-5 min, and emulsifying by the high-energy emulsification (high-speed shear at 10000-18000 rpm for 8-15 min) to obtain the primary emulsion W₁/O;
(5) preparing the external water phase W₂: adding 3 g of hydrophilic emulsifier (whey protein isolate here) to 97 g of deionized water, stirring for 2 hours to fully dissolve, and then putting in a 4° C. refrigerator overnight to fully hydrate, so as to obtain 3 wt% whey protein isolate solution, which is the external water phase W₂; and
(6) preparing the W₁/O/W₂ double emulsion: mixing the primary emulsionW₁/O and the external water phase W₂ at a mass ratio of 1:2, and emulsifying by the high-energy emulsification (high-speed shear at 10000-18000 rpm for 2-5 min) to obtain the W₁/O/W₂ double emulsion; storing at a low temperature of -20 to 4° C. to obtain the probiotic preparation.

2. Performance test:

using original probiotic solution and W₁/O/W₂ double emulsion without protective agent as a control group; using the Lactobacillus plantarum embedded in the double emulsion W₁/O/W₂ of the embodiment 10 as an experimental group, so as to observe the effects of protective agent on Lactobacillus plantarum viable count in the W₁/O/W₂ double emulsion; collecting equal amounts of appropriate samples before the control group and the experimental group are frozen at -18° C., after frozen at -18° C. for 12 hours, and after stored at 4° C. until completely thawed; determining the viable count by the dilution spread plate method.

Analysis results are shown in FIG. 12, it can be seen that the viable count of Lactobacillus plantarum decreases after freezing and thawing, and decrements vary for different samples. Viable count in the original probiotic solution before and after freezing and thawing is 4.30×10¹² cfu/mL and 4.26× 10⁹ cfu/mL, and the survival rate is 0.10%. Viable count in a product without protective agent before and after freezing and thawing is 4.72×10¹⁰ cfu/mL and 7.23×10⁹ cfu/mL, and the survival rate is 15.32%, while viable count in a product prepared in the embodiment 10 before and after freezing and thawing is 9.1×10¹⁰ cfu/mL and 5.18×10¹⁰ cfu/mL, and the survival rate is 56.92%. These data indicate that the W₁/O/W₂ double emulsion structure and the protective agent have a protective effect on Lactobacillus plantarum, indicating that the probiotic preparation of the present invention can inhibit the inactivation of the probiotics, so as to better exert their physiological activity in subsequent applications.

### Embodiment 11: Preparation of Double Emulsion Probiotic Preparation

1. A preparation method comprises steps of:

(1) preparing probiotic suspension: activating the probiotic Lactobacillus plantarum with an MRS broth medium, culturing in a constant temperature incubator at 30° C. for 14 hours, and then centrifuging probiotic liquid to obtain the probiotic;
(2) preparing the internal water phase W₁: preparing 10 wt% protective solution (trehalose here) as the probiotic protective agent, sterilizing at 121° C. for 15 min, and cooling to room temperature; mixing the probiotic with the probiotic protective agent, and dispersing evenly through vortex to form the internal water phase W₁;
(3) preparing the lipid phase O: adding 6 g of emulsifier (polyglycerol polyricinoleate here) to 94 g of oil (soybean oil here), and stirring magnetically at 500 rpm for 5-45 min to obtain 6 wt% polyglycerol polyricinoleate, namely the lipid phase O;
(4) preparing the primary emulsion W₁/O: dropping the internal water phase W₁ into the lipid phase O while stirring, wherein a mass ratio of the internal water phase W₁ to the lipid phase O is 1:3, a volume ratio of the internal water phase W₁ to the lipid phase O is 1:3, a stirring speed is 1000 rpm, and a dropping rate is 10-100 mL/min; then stirring for 2-5 min, and emulsifying by the high-energy emulsification (high-speed shear at 10000-18000 rpm for 8-15 min) to obtain the primary emulsion W₁/O;
(5) preparing the external water phase W₂: adding 0.1 g of hydrophilic emulsifier (whey protein isolate here) to 99.9 g of deionized water, stirring for 2 hours to fully dissolve, and then putting in a 4° C. refrigerator overnight to fully hydrate, so as to obtain 0.1 wt% whey protein isolate solution, which is the external water phase W₂; and
(6) preparing the W₁/O/W₂ double emulsion: mixing the primary emulsion W₁/O and the external water phase W₂ at a mass ratio of 1:2, and emulsifying by the high-energy emulsification (high-speed shear at 10000-18000 rpm for 2-5 min) to obtain the W₁/O/W₂ double emulsion; storing at a low temperature of -20 to 4° C. to obtain the probiotic preparation.

2. Performance test:

using original probiotic solution and W₁/O/W₂ double emulsion without protective agent as a control group; using the Lactobacillus plantarum embedded in the double emulsion W₁/O/W₂ of the embodiment 11 as an experimental group, so as to observe the effects of protective agent on Lactobacillus plantarum viable count in the W₁/O/W₂ double emulsion; collecting equal amounts of appropriate samples before the control group and the experimental group are frozen at -18° C., after frozen at -18° C. for 12 hours, and after stored at 4° C. until completely thawed; determining the viable count by the dilution spread plate method.

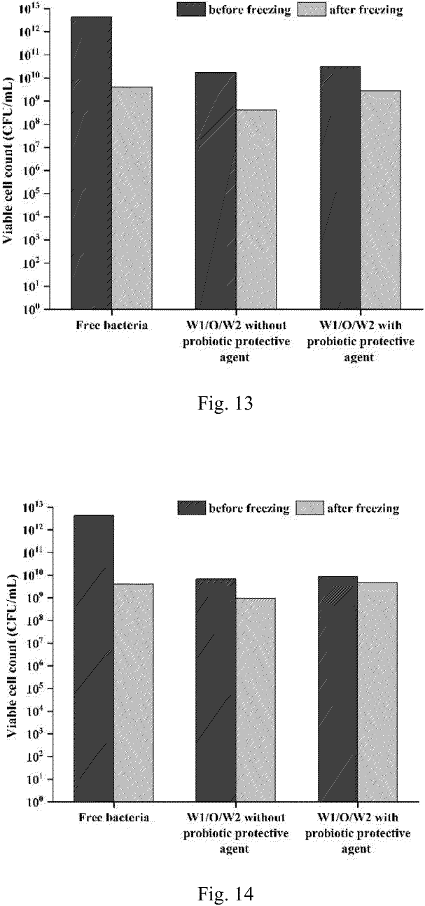

Analysis results are shown in FIG. 13, it can be seen that the viable count of Lactobacillus plantarum decreases after freezing and thawing, and decrements vary for different samples. Viable count in the original probiotic solution before and after freezing and thawing is 4.30×10¹² cfu/mL and 4.26× 10⁹ cfu/mL, and the survival rate is 0.10%. Viable count in a product without protective agent before and after freezing and thawing is 1.76×10¹⁰ cfu/mL and 4.31×10⁸ cfu/mL, and the survival rate is 2.45%, while viable count in a product prepared in the embodiment 11 before and after freezing and thawing is 3.11×10¹⁰ cfu/mL and 2.76×10⁹ cfu/mL, and the survival rate is 8.87%. These data indicate that the W₁/O/W₂ double emulsion structure and the protective agent have a protective effect on Lactobacillus plantarum, indicating that the probiotic preparation of the present invention can inhibit the inactivation of the probiotics, so as to better exert their physiological activity in subsequent applications.

### Embodiment 12: Preparation of Double Emulsion Probiotic Preparation

1. A preparation method comprises steps of:

(1) preparing probiotic suspension: activating the probiotic Lactobacillus plantarum with an MRS broth medium, culturing in a constant temperature incubator at 30° C. for 14 hours, and then centrifuging probiotic liquid to obtain the probiotic;
(2) preparing the internal water phase W₁: preparing 10 wt% protective solution (trehalose here) as the probiotic protective agent, sterilizing at 121° C. for 15 min, and cooling to room temperature; mixing the probiotic with the probiotic protective agent, and dispersing evenly through vortex to form the internal water phase W₁;
(3) preparing the lipid phase O: adding 6 g of emulsifier (polyglycerol polyricinoleate here) to 94 g of oil (soybean oil here), and stirring magnetically at 500 rpm for 5-45 min to obtain 6 wt% polyglycerol polyricinoleate, namely the lipid phase O;
(4) preparing the primary emulsion W₁/O: dropping the internal water phase W₁ into the lipid phase O while stirring, wherein a mass ratio of the internal water phase W₁ to the lipid phase O is 1:3, a stirring speed is 1000 rpm, and a dropping rate is 10-100 mL/min; then stirring for 2-5 min, and emulsifying by the high-energy emulsification (high-speed shear at 10000-18000 rpm for 8-15 min) to obtain the primary emulsion W₁/O;
(5) preparing the external water phase W₂: adding 3 g of hydrophilic emulsifier (whey protein isolate here) to 97 g of deionized water, stirring for 2 hours to fully dissolve, and then putting in a 4° C. refrigerator overnight to fully hydrate, so as to obtain 3 wt% whey protein isolate solution, which is the external water phase W₂; and
(6) preparing the W₁/O/W₂ double emulsion: mixing the primary emulsion W₁/O and the external water phase W₂ at a mass ratio of 1:99, and emulsifying by the high-energy emulsification (high-speed shear at 10000-18000 rpm for 2-5 min) to obtain the W₁/O/W₂ double emulsion; storing at a low temperature of -20 to 4° C. to obtain the probiotic preparation.

2. Performance test:

using original probiotic solution and W₁/O/W₂ double emulsion without protective agent as a control group; using the Lactobacillus plantarum embedded in the double emulsion W₁/O/W₂ of the embodiment 12 as an experimental group, so as to observe the effects of protective agent on Lactobacillus plantarum viable count in the W₁/O/W₂ double emulsion; collecting equal amounts of appropriate samples before the control group and the experimental group are frozen at -18° C., after frozen at -18° C. for 12 hours, and after stored at 4° C. until completely thawed; determining the viable count by the dilution spread plate method.

Analysis results are shown in FIG. 14, it can be seen that the viable count of Lactobacillus plantarum decreases after freezing and thawing, and decrements vary for different samples. Viable count in the original probiotic solution before and after freezing and thawing is 4.30×10¹² cfu/mL and 4.26× 10⁹ cfu/mL, and the survival rate is 0.10%. Viable count in a product without protective agent before and after freezing and thawing is 6.78×10⁹ cfu/mL and 9.58×10⁸ cfu/mL, and the survival rate is 14.13%, while viable count in a product prepared in the embodiment 12 before and after freezing and thawing is 8.95×10⁹ cfu/mL and 4.78×10⁹ cfu/mL, and the survival rate is 53.41%. These data indicate that the W₁/O/W₂ double emulsion structure and the protective agent have a protective effect on Lactobacillus plantarum, indicating that the probiotic preparation of the present invention can inhibit the inactivation of the probiotics, so as to better exert their physiological activity in subsequent applications.

### Embodiment 13: Preparation of Double Emulsion Probiotic Preparation

1. A preparation method comprises steps of:

(1) preparing probiotic suspension: activating the probiotic Lactobacillus plantarum with an MRS broth medium, culturing in a constant temperature incubator at 30° C. for 14 hours, and then centrifuging probiotic liquid to obtain the probiotic;
(2) preparing the internal water phase W₁: preparing 10 wt% protective solution (trehalose here) as the probiotic protective agent, sterilizing at 121° C. for 15 min, and cooling to room temperature; mixing the probiotic with the probiotic protective agent, and dispersing evenly through vortex to form the internal water phase W₁;
(3) preparing the emulsifier: mixing 3 g of polyglycerol polyricinoleate and 3 g of lecithin, and stirring magnetically at 500 rpm at 50° C. for 15-30 min, so as to obtain an emulsifier complex of an oil-soluble emulsifier and an amphiphilic emulsifier;
(4) preparing the lipid phase O: adding 6 g of emulsifier complex (the emulsifier complex of oil-soluble emulsifier polyglycerol polyricinoleate and amphiphilic emulsifier lecithin) to 94 g of oil (coconut oil here), and stirring magnetically at 500 rpm for 5-45 min to obtain 6 wt% emulsifier complex, namely the lipid phase O;
(5) preparing the primary emulsion W₁/O: dropping the internal water phase W₁ into the lipid phase O while stirring, wherein a mass ratio of the internal water phase W₁ to the lipid phase O is 1:3, a stirring speed is 1000 rpm, and a dropping rate is 10-100 mL/min; then stirring for 2-5 min, and emulsifying by the high-energy emulsification (high-speed shear at 10000-18000 rpm for 8-15 min) to obtain the primary emulsion W₁/O;
(6) preparing the external water phase W₂: adding 3 g of hydrophilic emulsifier (whey protein isolate here) to 97 g of deionized water, stirring for 2 hours to fully dissolve, and then putting in a 4° C. refrigerator overnight to fully hydrate, so as to obtain 3 wt% whey protein isolate solution, which is the external water phase W₂; and
(7) preparing the W₁/O/W₂ double emulsion: mixing the primary emulsion W₁/O and the external water phase W₂ at a mass ratio of 1:2, and emulsifying by the high-energy emulsification (high-speed shear at 10000-18000 rpm for 2-5 min) to obtain the W₁/O/W₂ double emulsion; storing at a low temperature of -20 to 4° C. to obtain the probiotic preparation.

2. Performance test:

using original probiotic solution and W₁/O/W₂ double emulsion without protective agent as a control group; using the Lactobacillus plantarum embedded in the double emulsion W₁/O/W₂ of the embodiment 13 as an experimental group, so as to observe the effects of protective agent on Lactobacillus plantarum viable count in the W₁/O/W₂ double emulsion; collecting equal amounts of appropriate samples before the control group and the experimental group are frozen at -18° C., after frozen at -18° C. for 12 hours, and after stored at 4° C. until completely thawed; determining the viable count by the dilution spread plate method.

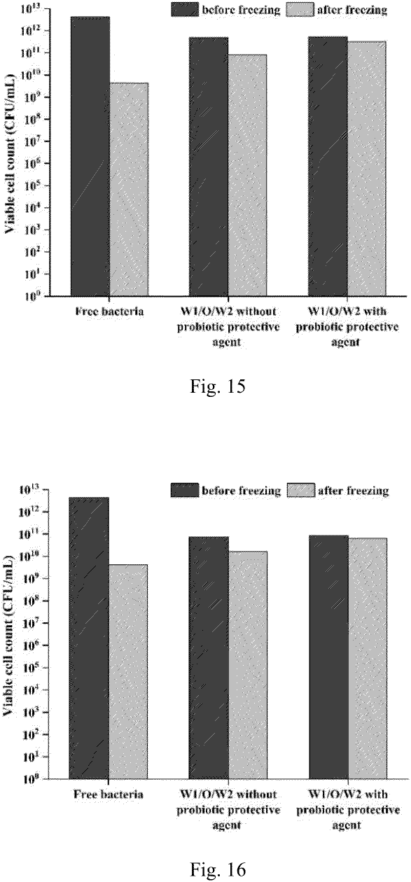

Analysis results are shown in FIG. 15, it can be seen that the viable count of Lactobacillus plantarum decreases after freezing and thawing, and decrements vary for different samples. Viable count in the original probiotic solution before and after freezing and thawing is 4.30×10¹² cfu/mL and 4.26× 10⁹ cfu/mL, and the survival rate is 0.10%. Viable count in a product without protective agent before and after freezing and thawing is 4.85×10¹¹ cfu/mL and 8.41×10¹⁰ cfu/mL, and the survival rate is 17.34%, while viable count in a product prepared in the embodiment 13 before and after freezing and thawing is 5.26×10¹¹ cfu/mL and 3.18×10¹¹ cfu/mL, and the survival rate is 60.46%. These data indicate that the W₁/OW₂ double emulsion structure and the protective agent have a protective effect on Lactobacillus plantarum, indicating that the probiotic preparation of the present invention can inhibit the inactivation of the probiotics, so as to better exert their physiological activity in subsequent applications.

### Embodiment 14: Preparation of Double Emulsion Probiotic Preparation

1. A preparation method comprises steps of:

(1) preparing probiotic suspension: activating the probiotic Lactobacillus plantarum with an MRS broth medium, culturing in a constant temperature incubator at 30° C. for 14 hours, and then centrifuging probiotic liquid to obtain the probiotic;
(2) preparing the internal water phase W₁: preparing 10 wt% protective solution (trehalose here) as the probiotic protective agent, sterilizing at 121° C. for 15 min, and cooling to room temperature; mixing the probiotic with the probiotic protective agent, and dispersing evenly through vortex to form the internal water phase W₁;
(3) preparing the lipid phase O: adding 6 g of emulsifier (polyglycerol polyricinoleate here) to 94 g of oil (soybean oil here), and stirring magnetically at 500 rpm for 5-45 min to obtain 6 wt% polyglycerol polyricinoleate, namely the lipid phase O;
(4) preparing the primary emulsion W₁/O: dropping the internal water phase W₁ into the lipid phase O while stirring, wherein a mass ratio of the internal water phase W₁ to the lipid phase O is 1:3, a stirring speed is 1000 rpm, and a dropping rate is 10-100 mL/min; then stirring for 2-5 min, and emulsifying by the membrane emulsification to obtain the primary emulsion W₁/O;
(5) preparing the external water phase W₂: adding 3 g of hydrophilic emulsifier (whey protein isolate here) to 97 g of deionized water, stirring for 2 hours to fully dissolve, and then putting in a 4° C. refrigerator overnight to fully hydrate, so as to obtain 3 wt% whey protein isolate solution, which is the external water phase W₂; and
(6) preparing the W₁/O/W₂ double emulsion: mixing the primary emulsion W1/O and the external water phase W₂ at a mass ratio of 1:2, and emulsifying by the membrane emulsification to obtain the W₁/O/W₂ double emulsion; storing at a low temperature of -20 to 4° C. to obtain the probiotic preparation.

2. Performance test:

using original probiotic solution and W₁/O/W₂ double emulsion without protective agent as a control group; using the Lactobacillus plantarum embedded in the double emulsion W₁/O/W₂ of the embodiment 14 as an experimental group, so as to observe the effects of protective agent on Lactobacillus plantarum viable count in the W₁/O/W₂ double emulsion; collecting equal amounts of appropriate samples before the control group and the experimental group are frozen at -18° C., after frozen at -18° C. for 12 hours, and after stored at 4° C. until completely thawed; determining the viable count by the dilution spread plate method.

Analysis results are shown in FIG. 16, it can be seen that the viable count of Lactobacillus plantarum decreases after freezing and thawing, and decrements vary for different samples. Viable count in the original probiotic solution before and after freezing and thawing is 4.30×10¹² cfu/mL and 4.26×10⁹ cfu/mL, and the survival rate is 0.10%. Viable count in a product without protective agent before and after freezing and thawing is 7.44×10¹⁰ cfu/mL and 1.61×10¹⁰ cfu/mL, and the survival rate is 21.64%, while viable count in a product prepared in the embodiment 14 before and after freezing and thawing is 8.24×10¹⁰ cfu/mL and 6.52×10¹⁰ cfu/mL, and the survival rate is 79.13%. These data indicate that the W₁/O/W₂ double emulsion structure and the protective agent have a protective effect on Lactobacillus plantarum, indicating that the probiotic preparation of the present invention can inhibit the inactivation of the probiotics, so as to better exert their physiological activity in subsequent applications.

### Embodiment 15: Preparation of Double Emulsion Probiotic Preparation

(1) preparing probiotic suspension: activating the probiotic Lactobacillus thermophiles with an MRS broth medium, culturing in a constant temperature incubator at 37° C. for 12-18 hours, and then centrifuging probiotic liquid to obtain the probiotic;
(2) preparing the internal water phase W₁: preparing a mixed solution containing 5 wt% sucrose, 0.5% NaCl, 0.01 wt% sodium glutamate, 5 wt% glycerol, 0.2 wt% polyethylene glycol 1000, 0.01 wt% alkaloid, and 20 wt% skimmed milk powder, sterilizing at 121° C. for 15 min, and cooling to room temperature; suspending the probiotic in the mixed solution to form the internal water phase W₁;
(3) preparing the lipid phase O: adding polyglycerol polyricinoleate tosoybean oil, and stirring magnetically at 500 rpm for 10-20 min to obtain 6 wt% polyglycerol polyricinoleate, namely the lipid phase O;
(4) preparing the primary emulsion W₁/O: dropping the internal water phase Wi into of the lipid phase O while stirring, wherein a mass ratio of the internal water phase Wi to the lipid phase O is 1:3, a stirring speed is 1000 rpm, and a dropping rate is 10-50 mL/min; then emulsifying by the high-pressure homogeneous emulsification to obtain the primary emulsion W₁/O;
(5) preparing the external water phase W₂: adding 3 g of whey protein isolate and 0.17 g of pectin to 96.83 g of deionized water, stirring for 2-3 hours to fully dissolve, and then putting in a 4° C. refrigerator overnight to fully hydrate, so as to obtain the external water phase W₂; and
(6) preparing the W₁/O/W₂ double emulsion: mixing the primary emulsion Wl/O and the external water phase W₂ at a mass ratio of 1:2, and emulsifying by the high-pressure homogeneous emulsification to obtain the W₁/O/W₂ double emulsion; storing at a low temperature of -20 to 4° C. to obtain the probiotic preparation.

2. Performance test:

using original probiotic solution and W₁/O/W₂ double emulsion without protective agent as a control group; using the Lactobacillus thermophiles embedded in the double emulsion W₁/O/W₂ of the embodiment 15 as an experimental group, so as to observe the effects of protective agent on Lactobacillus thermophiles viable count in the W₁/O/W₂ double emulsion; collecting equal amounts of appropriate samples before the control group and the experimental group are frozen at -18° C., after frozen at -18° C. for 12 hours, and after stored at 4° C. until completely thawed; determining the viable count by the dilution spread plate method.

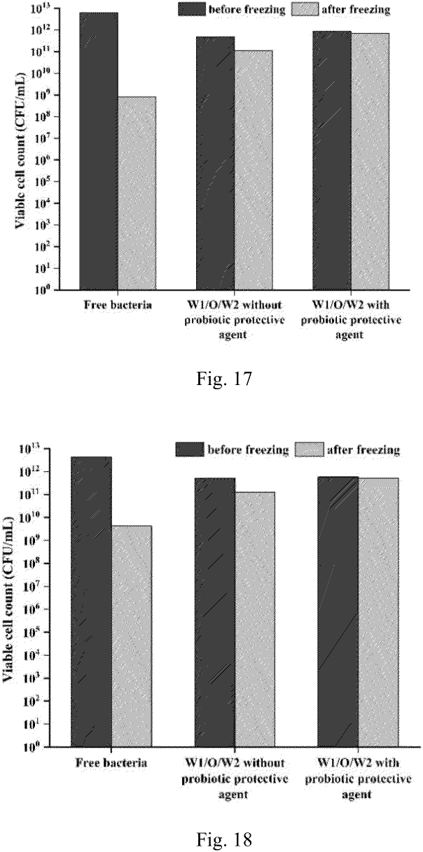

Analysis results are shown in FIG. 17, it can be seen that the viable count of Lactobacillus thermophiles decreases after freezing and thawing. Viable count in the original probiotic solution decreases from 6.36 × 10¹² cfu/mL and 8.43×10⁸ cfu/mL, and the survival rate is 0.01%. Viable count in a sample without protective agent before and after freezing and thawing is 4.79×10¹¹ cfu/mL and 1.11×10¹¹ cfu/mL, and the survival rate is 23.17%, while viable count in a sample prepared in the embodiment 15 before and after freezing and thawing is 8.52×10¹¹ cfu/mL and 6.87×10¹¹ cfu/mL, and the survival rate is 80.63%. These data indicate that the W₁/O/W₂ double emulsion structure and the complex protective agent (sucrose, glycerin and skim milk powder) have a good protective effect on Lactobacillus thermophiles, indicating that the probiotic preparation of the present invention can inhibit the inactivation of the probiotics, so as to better exert their physiological activity in subsequent applications.

### Embodiment 16: Preparation of Dual Emulsion Probiotic Preparation

1. A preparation method comprises steps of:

(1) preparing probiotic suspension: activating the probiotic Lactobacillus plantarum with an MRS broth medium, culturing in a constant temperature incubator at 30° C. for 14 hours, and then centrifuging probiotic liquid to obtain the probiotic;
(2) preparing the internal water phase W₁: preparing 10 wt% protective solution (trehalose here) as the probiotic protective agent, sterilizing at 121° C. for 10 min, and cooling to room temperature; mixing the probiotic with the probiotic protective agent, and dispersing evenly through vortex to form the internal water phase Wi;
(3) preparing the emulsifier: mixing 3 g of polyglyceryl polyricinoleate, 1 g of sucrose ester and 2 g of lecithin, and stirring magnetically at 500 rpm at 70° C. for 15-30 min, so as to obtain an emulsifier complex of an oil-soluble emulsifier and an amphiphilic emulsifier;
(4) preparing the lipid phase O: adding 6 g of emulsifier complex (the emulsifier complex of polyglyceryl polyricinoleate, lecithin and sucrose ester) to 94 g of oil (soybean oil here), and stirring magnetically at 500 rpm for 5-45 min to obtain the lipid phase O;
(5) preparing the primary emulsion W₁/O: dropping 5 g of the internal water phase Wi into 100 g of the lipid phase O while stirring, wherein a mass ratio of the internal water phase Wi to the lipid phase O is 1:3, a stirring speed is 1000 rpm, and a dropping rate is 10-100 mL/min; then stirring for 2-5 min, and emulsifying by the high-energy emulsification (high-speed shear at 10000-18000 rpm for 8-15 min) to obtain the primary emulsion W₁/O;
(6) preparing the external water phase W₂: adding 3 g of hydrophilic emulsifier (3 wt% whey protein isolate here) to 97 g of deionized water, stirring for 2 hours to fully dissolve, and then putting in a 4° C. refrigerator overnight to fully hydrate, so as to obtain 3 wt% hydrophilic emulsifier solution, which is the external water phase W₂; and
(7) preparing the W₁/O/W₂ dual emulsion: mixing the primary emulsion Wl/O and the external water phase W₂ at a mass ratio of 1:2, and emulsifying by the high-energy emulsification (high-speed shear at 10000-18000 rpm for 2-5 min) to obtain the W₁/O/W₂ dual emulsion; storing at a low temperature of -20 to 4° C. to obtain the probiotic preparation.

2. Performance test:

using original probiotic solution and W₁/O/W₂ dual emulsion without protective agent as a control group; using the Lactobacillus plantarum embedded in the dual emulsion W₁/O/W₂ of the embodiment 16 as an experimental group, so as to observe the effects of protective agent on Lactobacillus plantarum viable count in the W₁/O/W₂ dual emulsion; collecting equal amounts of appropriate samples before the control group and the experimental group are frozen at -18° C., after frozen at -18° C. for 12 hours, and after stored at 4° C. until completely thawed; determining the viable count by the dilution spread plate method.

Analysis results are shown in FIG. 18, it can be seen that the viable count of Lactobacillus plantarum decreases after freezing and thawing, and decrements vary for different samples. Viable count in the original probiotic solution before and after freezing and thawing is 4.30×10¹² cfu/mL and 4.26×10⁹ cfu/mL, and the survival rate is 0.10%. Viable count in a product without protective agent before and after freezing and thawing is 5.15×10¹¹ cfu/mL and 1.30×10¹¹ cfu/mL, and the survival rate is 25.24%, while viable count in a product prepared in the embodiment 16 before and after freezing and thawing is 5.73×10¹¹ cfu/mL and 5.25×10 ¹¹ cfu/mL, and the survival rate is 91.62%. These data indicate that the W₁/O/W₂ dual emulsion structure and the protective agent have a protective effect on Lactobacillus plantarum, indicating that the probiotic preparation of the present invention can inhibit the inactivation of the probiotics, so as to better exert their physiological activity in subsequent applications.

### Embodiment 17: Application of Double Emulsion Probiotic Preparation in Ice Cream

1. A method for preparing probiotic ice cream comprises steps of:

(1) mixing raw materials: weigh 10-15 wt% whole milk powder, 8-12 wt% white sugar, 8-15 wt% cream, 0.3-0.6 wt% compound stabilizer and water, and mixing evenly;
(2) performing sterilization and homogenization: pasteurizing at 90° C. for 20 min, and then cooling to about 60° C. for homogenization with homogenization pressure of 20.0 MPa;
(3) cooling and aging: after homogenization, quickly cooling the material to 2-4° C., adding 1-15 wt% of probiotic preparation which is embedded in the double emulsion prepared in the embodiment 1, and stirring to make the fat, protein and stabilizer fully expanded and combined, wherein an aging time is 4-8 h;
(4) freezing: adding fully aged ice cream slurry to a freezer for puffing, and mixing air into the ice cream slurry through freezing and whipping processes, to make volume-expanded soft ice cream;
(5) hardening: putting the soft ice cream into a mold, quick freezing at -20--25° C. in a refrigerator, and hardening for 12 h to make hard ice cream; and
(6) storing: the ice cream obtained after hardening is placed in a cold storage for preservation.

2. Quality inspection

Ice cream prepared from W₁/O/W₂ double emulsion without protective agent is used as a control group, and ice cream prepared from W₁/O/W₂ double emulsion with protective agent is used as an experimental group. After a certain period of time, proper samples of the control group and the experimental group are taken out of the cold storage. After thawing at room temperature, the viable count is measured respectively by the dilution spread plate method, thereby determining the effect of the protective agent and storage time on the viable count of the probiotic in the ice cream.

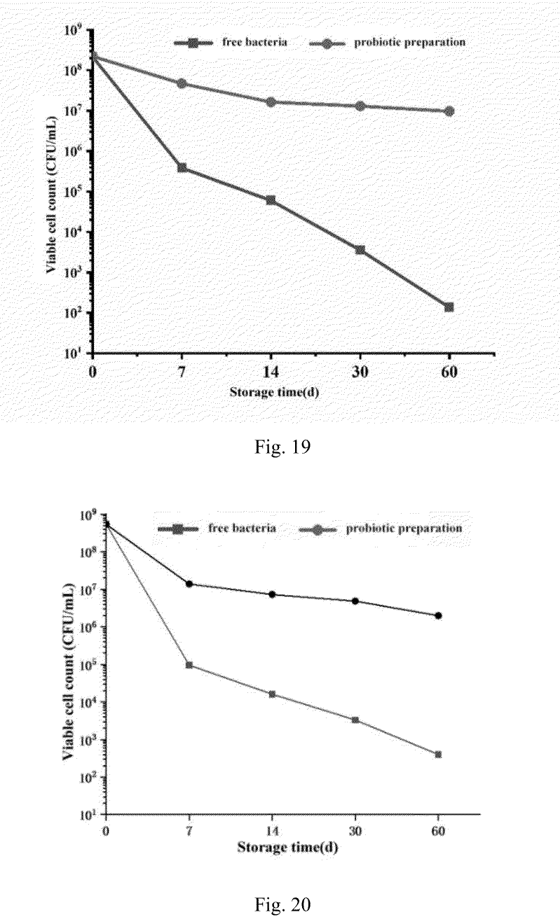

Results are shown in FIG. 19, it can be seen that in the ice cream without protective agent, the activity of Lactobacillus plantarum obviously drops over time, and activity loss rate is fast at first and then slow. The viable count of the ice cream with protective agent when the preparation is just completed is 2.21×10⁸ cfu/mL, and there is a slight loss in first 14 days. After that, the activity loss rate is significantly reduced, wherein the viable count of Lactobacillus plantarum is still 9.73×10⁶ cfu/mL on day 60, and survival rate of Lactobacillus plantarum is as high as 4.40%. The viable count of the ice cream without protective agent when the preparation is just completed is 3.40×10⁸ cfu/mL, and the viable count after 60 days is only 136.3 cfu/mL. This indicates that the protective agent has a significant protective effect on the activity of Lactobacillus plantarum during long-term storage, making it survive longer in ice cream and greatly prolonging its survival rate during the shelf life.

### Embodiment 18: Application of Double Emulsion Probiotic Preparation in Beverage

1. A method for preparing probiotic beverage comprises steps of:

(1) selecting and processing raw materials: selecting healthy and fresh sugarcane stems, washing and peeling the sugarcane, and squeezing juice; then filtering the juice with sterile gauze, and packaging into a tin;
(2) preparing a stabilizer: weighing 10-15 wt% whole milk powder, 8-12 wt% white sugar, 8-15 wt% cream, 0.3-0.6 wt% compound stabilizer and water, and mixing evenly;
(3) mixing the materials: adding the stabilizer to the sugarcane juice and thoroughly mixing;
(4) performing sterilization and homogenization: pasteurizing at 90° C. for 20 min, and then cooling to about 60° C. for homogenization with homogenization pressure of 20.0 MPa;
(5) cooling and aging: after homogenization, quickly cooling the material to 2-4° C., adding 1-15 wt% of probiotic preparation which is embedded in the double emulsion prepared in the embodiment 2, and stirring to make the double emulsion, sugarcane juice and stabilizer fully expanded and combined, wherein all processes are performed under sterile conditions; and
(6) storing: packaging the beverage in aseptic tins and storing in a 4° C. cold storage.

2. Quality inspection

Beverage prepared from W₁/O/W₂ double emulsion without protective agent is used as a control group, and beverage prepared from W₁/O/W₂ double emulsion with protective agent is used as an experimental group. After a certain period of time, proper samples of the control group and the experimental group are taken out of the cold storage. The viable count is measured respectively by the dilution spread plate method, thereby determining the effect of the protective agent and storage time on the viable count of the probiotic in the beverage.

Results are shown in FIG. 20, it can be seen that in the beverage without protective agent, the activity of Lactobacillus plantarum obviously drops over time. The viable count of the beverage with protective agent when the preparation is just completed is 5.38×10⁸ cfu/mL, wherein the viable count of Lactobacillus plantarum is still 1.99×10⁶ cfu/mL on day 60, and survival rate of Lactobacillus plantarum is up to 0.37%. The viable count of the beverage without protective agent when the preparation is just completed is 5.44×10⁸ cfu/mL, and the viable count after 60 days is only 401cfu/mL. This indicates that the protective agent has a significant protective effect on the activity of Lactobacillus plantarum during long-term storage, making it survive longer in beverage and greatly prolonging its survival rate during the shelf life.

### Embodiment 19: Application of Double Emulsion Probiotic Preparation in Animal Health Product

(1) preparing a glucose solution: dissolving glucose in 0.6 wt% physiological saline to prepare a 15 wt% glucose solution, and dividing the solution into two parts of 1:3;
(2) preparing vitamin water emulsion solution: mixing 5×10⁶IU vitamin A, 1.5×10⁶IU vitamin D3 and 0.5×10⁴IU vitamin E evenly, then adding 0.6×10³ mg tyrosine and thoroughly mixing; then adding 8 wt% lecithin and 10 wt% sorbitol, and dispersing by shearing at 55° C. for 25 min under a rotational speed of 10,000 rpm; after cooling, adding the emulsion to a smaller portion of the glucose solution and stirred evenly to obtain a vitamin water emulsion solution;
(3) preparing vitamin/amino acid mixed solution: adding 2.4×10³IU vitamin B1, 1.8×10³IU vitamin B6, 5×10³IU vitamin B12, 0.6×10⁴ mg nicotinamide and 9 mg biotin to another portion of the glucose solution, and stirring at 50° C. for completely dissolving; then adding 1.4 ×10³ mg lysine, 0.6×10³ mg methionine, 1.2 ×10³ mg aspartic acid, 0.6 ×10³ mg histidine, 2.3×10³ mg glycine, 0.8×10³ mg leucine and 0.7 ×10³ mg isoleucine, and stirring for completely dissolving, so as to obtain the vitamin/amino acid mixed solution; and
(4) preparing an animal health care product: adding the vitamin water emulsion solution into the vitamin/amino acid mixed solution while stirring, and keeping temperature at 20° C.; then adding 3.8 wt% of the probiotic preparation prepared in the embodiment 2 and mixing evenly; and storing in a 4° C. refrigerator to obtain the animal health care product.

2. Quality inspection

Animal health product prepared from W₁/O/W₂ double emulsion without protective agent is used as a control group, and animal health product prepared from W₁/O/W₂ double emulsion with protective agent is used as an experimental group. After a certain period of time, proper samples of the control group and the experimental group are taken out of the cold storage. The viable count is measured respectively by the dilution spread plate method, thereby determining the effect of the protective agent and storage time on the viable count of the probiotic in the animal health product.

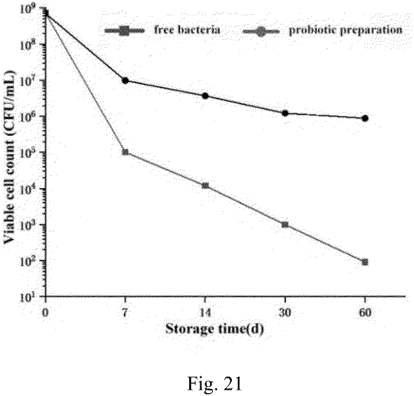

Results are shown in FIG. 21 it can be seen that in the animal health product without protective agent, the activity of Lactobacillus plantarum obviously drops over time. The viable count of the animal health product with protective agent when the preparation is just completed is 7.11×10⁸ cfu/mL, wherein the viable count of Lactobacillus plantarum is still 8.79×10⁵ cfu/mL on day 60, and survival rate of Lactobacillus plantarum is up to 0.12%. The viable count of the animal health product without protective agent when the preparation is just completed is 7.43×10⁸ cfu/mL, and the viable count after 60 days is only 92 cfu/mL. This indicates that the protective agent has a significant protective effect on the activity of Lactobacillus plantarum during long-term storage, making it survive longer in animal health product and greatly prolonging its survival rate during the shelf life.

The above embodiments are only preferred embodiments of the present invention, and are not intended to be limiting. Any modifications, equivalent replacements and improvements made within the spirit and principles of the present invention should be included in the protection scope of the present invention.

What is claimed is:

1. A probiotic preparation based on a W₁/O/W₂ double emulsion structure, comprising a double emulsion, wherein the double emulsion comprises a lipid phase O and a water phase W₂, and the lipid phase O is distributed inside the water phase W₂; the lipid phase O contains a plurality of water droplets W₁, and W₁ is a solution containing probiotics or a mixed solution of probiotics and a probiotic protective agent; the water droplets W₁ are stabilized inside the lipid phase O by emulsifiers.

2. A method for preparing a probiotic preparation based on a W₁/O/W₂ double emulsion structure, comprising steps of:
S1: processing probiotics or a mixture of probiotics and a probiotic protective agent with a vortex treatment, so as to evenly disperse and form an internal water phase W₁;
S2: preliminarily mixing a lipid phase O with the internal water phase W₁, and stabilizing with an emulsifier; then preparing a water-in-oil primary emulsion W₁/O by stirring and low-energy emulsification or high-energy emulsification, thereby stabilizing the W₁ is in the lipid phase O; and
S3: using an emulsifier-contained solution as an external water phase W₂, and adding the external water phase W₂ to the primary emulsion W₁/O; then preparing a W₁/O/W₂ double emulsion by stirring, the low-energy emulsification or the high-energy emulsification, thereby obtaining the probiotic preparation.

3. The method, as recited in claim 2, wherein:
in the step S1, the internal water phase W₁ contains the probiotics or the mixture of the probiotics and the probiotic protective agent;
the probiotic protective agent is selected from a group consisting of carbohydrates, proteins, amino acid salts, alcohols, inorganic salts, antioxidants, alkaloids, polymers and complexes; a concentration of the probiotic protective agent in the internal water phase is more than 0.01 wt%;
the probiotic protective agent is selected from a group consisting of glycerin, skimmed milk powder, ascorbic acid, whey protein isolate, trehalose, sucrose, fructose, maltose, lactose, dextran, gelatin, peptone, methylcellulose, betaine, sodium glutamate, xylitol, polyethylene glycol 1000 and sodium dodecyl sulfonate.

4. The method, as recited in claim 2, wherein:
in the step S2, the low-energy emulsification contains phase transition, spontaneous emulsification, or membrane emulsification; the high-energy emulsification contains high-speed shear, high-pressure homogeneous emulsification, or microfluidic emulsification.

5. The method, as recited in claim 2, wherein:
an adding amount of the emulsifier is at least 0.1 wt% of the lipid phase O; and the lipid phase is at most 90 wt% of a total emulsion weight;
the emulsifier is selected form a group consisting of an oil-soluble emulsifier, a hydrophilic emulsifier, and an amphiphilic emulsifier;
the oil-soluble emulsifier is selected form a group consisting of polyglycerol polyricinoleate (PGPR), Span20, Span60, Span65, Span80, Span85, ethylene glycol fatty acid ester, propylene glycol monostearate ester, glyceryl monostearate, and zein; the hydrophilic emulsifier is selected form a group consisting of Tween 80, pectin, hydroxypropyl methylcellulose, whey protein isolate, whey protein concentrate, casein, and β-lactoglobulin fiber; the amphiphilic emulsifier is selected form a group consisting of lecithin and sucrose ester;
the lipid phase is selected from a group consisting of fat, sterol, and lipoid which are liquid at ambient temperature, and from a group consisting of fat, sterol, and lipoid that are solid at ambient temperature;
the liquid fat is modified or unmodified vegetable and animal oils, which is selected from a group consisting of palm oil, avocado oil, mustard oil, linseed oil, grape oil, peanut oil, coconut oil, olive oil, thistle oil, grapeseed oil, sesame oil, soybean oil, sunflower oil, flaxseed oil, cotton oil, rapeseed oil, low-erucic acid rapeseed oil, corn oil, rice oil, safflower oil, kapok oil, sesame oil, evening primrose oil, fish oil, and seafood oil; the solid fat is modified or unmodified vegetable and animal oils, which is selected from a group consisting of cocoa butter, shea butter, sal resin, chicken fat, tallow, milk fat, and lard; the sterol and the lipoid are selected from a group consisting of modified or unmodified beeswax, paraffin wax, sunflower wax, rice wax, candelilla wax, palm wax and sitosterol; modification refers to hydrogenation, fractionation and/or transesterification.

6. The method, as recited in claim 2, wherein the low-energy emulsification is phase transition, spontaneous emulsification, or membrane emulsification; and the high-energy emulsification is high-speed shear, high-pressure homogeneous emulsification, or microfluidic emulsification.

7. The method, as recited in claim 2, wherein in the external water phase W₂, a mass percentage of the emulsifier is at least 0.1 wt%; a volume ratio of the external water phase W₂ to the primary emulsion W₁/O is at most 99%;
the emulsifier is selected from a group consisting of small molecule emulsifiers, polysaccharide emulsifiers, polypeptide emulsifiers, protein emulsifiers, polymers and complexes;
the emulsifier is selected from a group consisting of Tween 80, pectin, hydroxypropyl methylcellulose, whey protein isolate, whey protein concentrate, casein, and β-lactoglobulin fiber.

8. The method, as recited in claim 2, wherein the lipid phase O in the step S2 is formed by dissolving polyglycerol polyricinoleate and lecithin in edible oil.

9. The method, as recited in claim 8, wherein in the step S2, a method for preparing the lipid phase O comprises specific steps of:
(1) preparing the emulsifier: mixing 3 g of polyglycerol polyricinoleate and 3 g of lecithin, and stirring magnetically at 500 rpm at 50° C. for 15-30 min, so as to obtain an emulsifier complex of an oil-soluble emulsifier and an amphiphilic emulsifier; and
(2) preparing the lipid phase O: adding 6 g of the emulsifier complex prepared in the step (1) to 94 g of soybean oil, and stirring magnetically at 500 rpm for 5-45 min to obtain the lipid phase O.

10. The method, as recited in claim 2, wherein the lipid phase O in the step S2 is formed by dissolving polyglycerol polyricinoleate, lecithin, and sucrose ester in edible oil.

11. The method, as recited in claim 10, wherein in the step S2, a method for preparing the lipid phase O comprises specific steps of:
(1) preparing the emulsifier: mixing 3 g of polyglycerol polyricinoleate, 1 g of sucrose ester and 2 g of lecithin, and stirring magnetically at 500 rpm at 70° C. for 15-30 min, so as to obtain an emulsifier complex of an oil-soluble emulsifier and an amphiphilic emulsifier; and
(2) preparing the lipid phase O: adding 6 g of the emulsifier complex prepared in the step (1) to 94 g of soybean oil, and stirring magnetically at 500 rpm for 5-45 min to obtain the lipid phase O.

12. The method, as recited in claim 2, comprising specific steps of:
(1) preparing probiotic suspension: activating the probiotic Lactobacillus plantarum with an MRS broth medium, culturing in a constant temperature incubator at 30° C. for 14 hours, and then centrifuging probiotic liquid to obtain the probiotic;
(2) preparing the internal water phase W₁: preparing 10 wt% trehalose solution as the probiotic protective agent, sterilizing at 121° C. for 15 min, and cooling to room temperature; mixing the probiotic with the probiotic protective agent, and dispersing evenly through vortex to form the internal water phase W₁;
(3) preparing the emulsifier: mixing 3 g of polyglycerol polyricinoleate and 3 g of lecithin, and stirring magnetically at 500 rpm at 50° C. for 15-30 min, so as to obtain an emulsifier complex of an oil-soluble emulsifier and an amphiphilic emulsifier;
(4) preparing the lipid phase O: adding 6 g of the emulsifier complex prepared in the step (3) to 94 g of soybean oil, and stirring magnetically at 500 rpm for 5-45 min to obtain the lipid phase O
(5) preparing the primary emulsion W₁/O: dropping the internal water phase W₁ into the lipid phase O while stirring, wherein a mass ratio of the internal water phase W₁ to the lipid phase O is 1:3, a stirring speed is 1000 rpm, and a dropping rate is 10-100 mL/min; then stirring for 2-5 min, and emulsifying by the high-energy emulsification to obtain the primary emulsion W₁/O;
(6) preparing the external water phase W₂: adding 3 g of whey protein isolate to 97 g of deionized water, stirring for 2 hours to fully dissolve, and then putting in a 4° C. refrigerator overnight to fully hydrate, so as to obtain 3 wt% whey protein isolate solution, which is the external water phase W₂; and
(7) preparing the W₁/O/W₂ double emulsion: mixing the primary emulsion W₁/O and the external water phase W₂ at a mass ratio of 1:2, and emulsifying by the high-energy emulsification to obtain the W₁/O/W₂ double emulsion; storing at a low temperature of -20 to 4° C. to obtain the probiotic preparation.

13. The method, as recited in claim 2, comprising specific steps of:
(1) preparing probiotic suspension: activating the probiotic Lactobacillus plantarum with an MRS broth medium, culturing in a constant temperature incubator at 30° C. for 14 hours, and then centrifuging probiotic liquid to obtain the probiotic;
(2) preparing the internal water phase W₁: preparing 10 wt% protective solution as the probiotic protective agent, sterilizing at 121° C. for 10 min, and cooling to room temperature; mixing the probiotic with the probiotic protective agent, and dispersing evenly through vortex to form the internal water phase W₁;
(3) preparing the emulsifier: mixing 3 g of polyglycerol polyricinoleate, 1 g of sucrose ester and 2 g of lecithin, and stirring magnetically at 500 rpm at 70° C. for 15-30 min, so as to obtain an emulsifier complex of an oil-soluble emulsifier and an amphiphilic emulsifier;
(4) preparing the lipid phase O: adding 6 g of the emulsifier complex prepared in the step (3) to 94 g of soybean oil, and stirring magnetically at 500 rpm for 5-45 min to obtain the lipid phase O
(5) preparing the primary emulsion W₁/O: dropping the internal water phase W₁ into the lipid phase O while stirring, wherein a mass ratio of the internal water phase W₁ to the lipid phase O is 1:3, a stirring speed is 1000 rpm, and a dropping rate is 10-100 mL/min; then stirring for 2-5 min, and emulsifying by the high-energy emulsification to obtain the primary emulsion W₁/O;
(6) preparing the external water phase W₂: adding 3 g of hydrophilic emulsifier to 97 g of deionized water, stirring for 2 hours to fully dissolve, and then putting in a 4° C. refrigerator overnight to fully hydrate, so as to obtain 3 wt% hydrophilic emulsifier solution, which is the external water phase W₂; and
(7) preparing the W₁/O/W₂ double emulsion: mixing the primary emulsion W₁/O and the external water phase W₂ at a mass ratio of 1:2, and emulsifying by the high-energy emulsification to obtain the W₁/O/W₂ double emulsion; storing at a low temperature of -20 to 4° C. to obtain the probiotic preparation.
